(19) United States
(12) Patent Application Publication
(10) Pub. No.: US 2021/0386790 A1
(43) Pub. Date: Dec. 16, 2021

(54) USE OF MESENCHYMAL STEM CELL SHEETS FOR PREVENTING UTERINE SCAR FORMATION

(71) Applicant: University of Utah Research Foundation, Salt Lake City, UT (US)

(72) Inventors: Goro Kuramoto, Salt Lake City, UT (US); Teruo Okano, Salt Lake City, UT (US); Robert Silver, Salt Lake City, UT (US); David Grainger, Salt Lake City, UT (US)

(73) Assignee: University of Utah Research Foundation, Salt Lake City, UT (US)

(21) Appl. No.: 17/422,646

(22) PCT Filed: Jan. 15, 2020
(86) PCT No.: PCT/US2020/013622

§ 371 Date: Jul. 13, 2021

Related U.S. Application Data
(60) Provisional application No. 62/793,195, filed on Jan. 16, 2019.

(51) Int. Cl. A61K 35/28; A61K 35/51; A61L 27/38

(52) U.S. Cl. CPC A61K 35/28; A61L 2430/22; A61L 27/3834; A61K 35/51

(57) ABSTRACT

The disclosure provides a method of reducing formation of fibrotic tissue in a uterus of a subject in need thereof, comprising applying a mesenchymal stem cell (MSC) sheet to the uterus of the subject, wherein the MSC sheet comprises one or more layers of aggregated confluent mesenchymal stem cells (MSCs), and wherein applying the MSC sheet to the uterus reduces the formation of fibrotic tissue in the uterus relative to a uterus in which the MSC sheet is not applied. The disclosure also provides a method of increasing myometrial regeneration in a uterus of a subject in need thereof, comprising applying a mesenchymal stem cell (MSC) sheet to the uterus of the subject, wherein the MSC sheet comprises one or more layers of aggregated confluent mesenchymal stem cells (MSCs), and wherein applying the MSC sheet to the uterus increases myometrial regeneration relative to a uterus in which the MSC sheet is not applied.

### RELATED APPLICATIONS

This application claims priority to U.S. Provisional Patent Application No. 62/793,195 filed on Jan. 16, 2019, the contents of which are incorporated herein in their entirety.

### BACKGROUND OF THE INVENTION

Uterine scar development after Caesarean Delivery and other uterine surgeries can cause complications such as uterine rupture and abnormal placentation. These complications can result in a very serious prognosis for both the mother and the fetus. A clinical study reported that Caesarean Delivery should be performed very quickly (e.g. within 18 minutes) after diagnosis of a fetal disorder due to uterine rupture for preventing a higher incidence of perinatal mortality and morbidity. See Leung et al., 1993, Am J Obstet Gynecol 169: 945-950. The risk of these complications can increase depending on the number of times a Caesarean Delivery is performed. In particular, women having multiple Caesarean Deliveries could have higher risk. Therefore, a need exists to prevent potential complications resulting from Caesarean Deliveries and other uterine surgeries.

### SUMMARY OF THE INVENTION

In certain aspects, the present disclosure relates to a method of reducing formation of fibrotic tissue in a uterus of a subject in need thereof, comprising applying a mesenchymal stem cell (MSC) sheet to the uterus of the subject, wherein the MSC sheet comprises one or more layers of aggregated confluent mesenchymal stem cells (MSCs), and wherein applying the MSC sheet to the uterus reduces the formation of fibrotic tissue in the uterus relative to a uterus in which the MSC sheet is not applied.

In certain aspects, the present disclosure relates to a method of increasing myometrial regeneration in a uterus of a subject in need thereof, comprising applying a mesenchymal stem cell (MSC) sheet to the uterus of the subject, wherein the MSC sheet comprises one or more layers of aggregated confluent mesenchymal stem cells (MSCs), and wherein applying the MSC sheet to the uterus increases myometrial regeneration relative to a uterus in which the MSC sheet is not applied.

In certain aspects, the present disclosure relates to a method of preventing or reducing rupture of a uterine incision and abnormal placentation in a subject in need thereof, comprising applying a mesenchymal stem cell (MSC) sheet to the uterus of the subject, wherein the MSC sheet comprises one or more layers of aggregated confluent mesenchymal stem cells (MSCs), and wherein applying the MSC sheet to the uterus prevents or reduces rupture of the uterine incision and abnormal placentation relative to an incision in a uterus to which the MSC sheet is not applied.

In some embodiments, the MSC sheet is applied to an incision site in the uterus. In some embodiments, applying the MSC sheet to the uterus reduces fibrotic surface area of the uterus by at least 20% relative to a uterus in which the MSC sheet is not applied. In some embodiments, the MSC sheet consists essentially of MSCs. In some embodiments, the cell sheet comprises an extracellular matrix. In some embodiments, the extracellular matrix comprises one or more proteins selected from the group consisting of fibronectin, laminin and collagen. In some embodiments, the cell sheet comprises cell adhesion proteins and cell junction proteins. In some embodiments, the cell junction proteins are selected from the group consisting of Vinculin, Integrin-β1, Connexin 43, β-catenin, Integrin-linked kinase and N-cadherin. In some embodiments, the MSCs are isolated from the subepithelial layer of human umbilical cord tissue. In some embodiments, the MSCs express a protein selected from CD44 and CD90. In some embodiments, the MSCs express a cytokine selected from the group consisting of hepatocyte growth factor (HGF), vascular endothelial growth factor (VEGF) and interleukin-10 (IL-10). In some embodiments, expression of the cytokine in the cell sheet is increased relative to a suspension of cultured, isolated MSCs containing an equivalent number of cells. In some embodiments, the cell sheet expresses the cytokine for at least 10 days after transplantation to a tissue in a host organism.

In some embodiments, the cell sheet expresses extracellular matrix proteins and cell junction proteins for at least 10 days after transplantation to a tissue in a host organism. In some embodiments, the extracellular matrix proteins are selected from the group consisting of fibronectin, laminin and collagen. In some embodiments, the cell junction proteins are selected from the group consisting of Vinculin, Integrin-β1, Connexin 43, β-catenin, Integrin-linked kinase and N-cadherin. In some embodiments, initial cell density of the MSCs in a cell culture support used to prepare the cell sheet is from 0.5×10⁴/cm² to 9×10⁵/cm². In some embodiments, the MSCs do not express Human Leukocyte Antigen-DR isotype (HLA-DR), Human Leukocyte Antigen-DP isotype (HLA-DP), or Human Leukocyte Antigen-DQ isotype (HLA-DQ). In some embodiments, the MSCs comprise microvilli and filopodia.

In some embodiments, the cell sheet remains attached to a tissue in a host organism for at least 10 days after transplantation to the tissue.

In some embodiments, the MSCs in the cell sheet are allogeneic to the subject. In some embodiments, the subject is a human. In some embodiments, the subject has had at least one Caesarean Delivery. In some embodiments, the subject has had at least two Caesarean Deliveries. In some embodiments, the subject has had at least one uterine surgery. In some embodiments, the MSC is a human umbilical cord mesenchymal stem cell (hUC-MSC).

### BRIEF DESCRIPTION OF THE DRAWINGS

FIG. 1 shows the cell sheet experimental protocol. Human umbilical cord stem cells (hUC-MSCs) were seeded on temperature responsive cell culture dishes (TRCD) and cultured to confluence in a 37° C. cell culture incubator. Cultured cells were detached from TRCDs as intact cell sheets within 20 min. at room temperature (RT), preserving essential features including extracellular matrices (ECMs) and cell-cell-junctions without using proteolytic enzyme treatments.

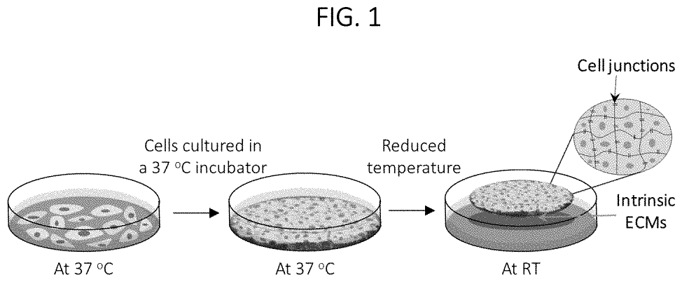

FIG. 2A-2B shows hUC-MSC sheet morphological observations using cell passages 4, 6, 8, 10 and 12 seeded at 2×10⁴ cells/cm². (a) Morphology of passage 4, 6, 8, 10 and 12 cells observed using phase-contrast microscopy before sheet detachment. (b) Successful fabrication of hUC-MSC sheets using passage 4, 6, 8 and 10 cells. In contrast, passage 12 cells detached as non-contiguous disconnected cellular structures. Scale bars=100 μm.

FIG. 3A-3C shows morphological observation, cell proliferation rate, and cell sheet fabrication for hUC-MSCs seeded at 2×10⁵, 1×10⁵ and 5×10⁴ initial cell numbers on a 35 mm diameter TRCD with a surface area of 9.6 cm². (a) Cells cultured on TRCD and observed prior to sheet detachment. (b) Cell numbers counted using a hemocytometer after cells were seeded on TRCD until hUC-MSC sheets are observed. (c) Cells detached as disconnected fragments at one day prior to confluence at 3, 4 and 5 days in 2×10⁵, 1×10⁵ and 5×10⁴ initial cell seeded groups, respectively. Intact cell sheets were successfully fabricated at 4, 5 and 6 day for seeding densities of 2×10⁵, 1×10⁵ and 5×10⁴ initial cell number groups, respectively. One day post-confluence, cultured cells spontaneously detach as aggregated fragments without TRCD temperature changes at 5, 6 and 7 days for the 2×10⁵, 1×10⁵ and 5×10⁴ initial cell seeded groups, respectively. Scale bars indicate 100 μm in (a). Scale bars indicate 1 cm in (c).

FIG. 4A-4D shows CD44 and CD90 positive expression in hUC-MSCs in cell suspension cultures (A and B) and in hUC-MSC sheets in vitro (C and D).

FIG. 5A-5E shows cell-cell structural analysis using immunohistochemistry (IHC) and transmission electron microscopy (TEM). Cultured cells successfully detached as sheets from TRCD by temperature changes at 4 days after seeding. Cell sheets were stained (bright areas SA-C) with ECM ((a) fibronectin and (b) laminin), and (c) cell junction β-catenin) antibodies, respectively, to confirm that cell sheets preserved their functional structures after detachment. In TEM images, the hUC-MSC sheet preserved their (d) ECMs and (e) cell-cell junction structures after detachment. 5D arrow=ECMs; 5E arrow=cell junctions in the hUC-MSC sheet. Scale bars indicate 100 μm in (a-c). Scale bars (d) and (e) indicate 5 μm and 1 μm, respectively.

FIG. 6A-6D shows cytokine analysis of human hepatocyte growth factor (HGF) and tumor necrosis factor-alpha (TNF-α) secreted from hUC-MSC sheets. hHGF (anti-inflammatory cytokine) and hTNF-α (pro-inflammatory cytokine) were detected in culture supernatant for cells cultured for 24 hours. (a) No significant differences in hHGF secretion in 2×10⁵, 1×10⁵, and 5×10⁴ initial cell seeded groups. (b); hTNF-α barely detected and not significantly different in 2×10⁵, 1×10⁵, and 5×10⁴ initial cell seeded groups. (c) Significant reductions in hHGF secreted from hUC-MSC sheets as passage increases. (d) hUC-MSC sheet fabricated using passage 4 cells secreted significantly lower amount of hTNF-α, compared to hUC-MSC sheet fabricated using passage 6, 8, 10, and 12 cells *p<0.05

FIG. 7A-7E shows implanted hUC-MSC sheet retention in vivo. hUC-MSC sheets implanted within subcutaneous tissue in immuno-deficient mice. (c and d) At 10 days after implantation, hUC-MSC transplanted subcutaneous tissue sites were harvested for histological observation. In H&E-stained images, (b) the hUC-MSC cell sheet was confirmed clearly in subcutaneous tissue implant sites compared to (a) normal subcutaneous tissue (control). Additionally, (e) abundant vascular structures are observed in cell sheet implanted groups. Arrows in (b)=implanted cell sheet; arrows in (e)=blood vessels. Scale bars (a and b) and (e) indicate 100 μm and 50 μm, respectively. Scale bars (c and d) indicated 0.5 cm.

FIG. 8A-8B shows cell-cell junction related gene expression levels from hUC-MSC sheets. Gene expression levels of (a) integrin-linked protein kinase (ILK) and (b) N-cadherin (Ncad) associated with cell junctions in passage 12 were lower than that in passage 6.

FIG. 9A-9C shows an illustration of the cell harvesting process. Human umbilical cord mesenchymal stem cells (hUC-MSC) were seeded on a 35 mm temperature responsive cell culture dish (TRCD) or tissue culture plate (TCP) and cultured for 5 days to reach confluence. hUC-MSC were harvested using 3 different methods which represents cell sheet technology, chemical disruption and physical disruption. A) Cell sheet is harvested contiguously and intact by temperature change, B) cells are treated with enzymatic proteolysis (trypsin), yielding single cell suspensions and cell aggregates; C) cells are harvested physically using a cell scraper, yielding heterogeneous multi-cell fragments and aggregates.

FIG. 10A-10D shows preparation of human umbilical cord mesenchymal stem cells (hUC-MSC) sheet (A) cells were cultured on conventional tissue culture plate (TCP) or temperature responsive cell culture dish (TRCD) for 5 days. Cell morphologies cultured on TCP and TRCD were observed using phase contrast microscope. (B) Cell number was counted using hemocytometer when they are cultured on TCP or TRCD for 100 hours. (C) The cells cultured on TRCD were detached as a sheet form by temperature reduction. (D) Histological analysis of the harvested cell sheet was performed by H&E staining to show individual cell bodies and nuclei within sheet forms. Scale bars indicate 200 μm in A and D, and 10 mm in C.

FIG. 11A-11H distinguishes different morphological observations of hUC-MSC cells harvested by trypsin proteolysis and hUC-MSC sheets harvested using temperature changes without enzymes. (A) Morphology of trypsinized MSC surface observed using scanning electron microscopy (SEM). (B) Microstructures of temperature harvested hUC-MSC sheets and trypsinized hUC-MSCs analyzed using transmission electron microscope (TEM). White arrows in (E) indicate intact MSC sheet cell junctions, dark grey arrows in (H) indicate intact hUC-MSC sheet ECM. Scale bar=5 μm in SEM and TEM.

FIG. 12A-12C shows cell dynamic-related protein expression analysis using western blot and immunohistochemistry for temperature-harvested hUC-MSC cell sheets compared to trypsinized hUC-MSCs. (A) Western blot of F-actin, Vinculin and GAPDH in intact cells sheets (left lane) compared to trypsinized cells (whole cell lysates, 10 mg protein/lane). Immunostaining fluorescence imaging comparisons of intact hUC-MSC cell sheets (left images) compared to trypsinized hUC-MSC harvests (right lanes) for (B) F-actin cytoskeleton, (C) vinculin and nuclear DAPI (bright punctate spots). Scale bar=10 μm.

FIG. 13A-13C shows ECM protein expression comparisons using western blot and immunohistochemistry fluorescence imaging of intact hUC-MSC cell sheets (left images) compared to trypsinized hUC-MSC harvests (right images) for. (A) Western blots of fibronectin, laminin and GAPDH in whole cell lysates (10 mg protein/lane). Immunostaining of (B) fibronectin, (C) laminin and nucelar DAPI (bright spots). Scale bar=10 μm.

FIG. 14A-14C shows cell-ECM and cell-cell junction protein expression comparisons using western blot and immunohistochemistry fluorescence imaging comparisons of intact hUC-MSC cell sheets (left images) compared to trypsinized hUC-MSC harvests (right lanes) for (A) western blots of Integrin β-1, Connexin 43 and GAPDH in whole cell lysates (10 mg protein/lane). Immunostaining images of (B) Integrin β-1, (C) Connexin 43, and DAPI (bright spots). Scale bar=10 μm.

FIG. 15A-15C compares living and dead cells in intact hUC-MSC cell sheets versus trypsinized hUC-MSC suspensions using dye-based assay: (A) microscopic images of live and dead staining of cell sheets (left side) and trypsinized cell suspensions (right lanes). Cells were stained by calcein and ethidium homodimer-1 immediately after cell detachment. Scale bar=100 μm; (B) quantitation of live and dead cells for cell sheets compared to trypsinized hUC-MSCs from image analysis; (C) data for live/dead cell quantitation for graph shown in (B).

FIG. 16 compares protein mechanosensor expression analysis using western blots of hUC-MSC cell sheets versus trypsinized hUC-MSC suspensions. Western blot of Yes associated protein (YAP), phosphorylated-YAP and GAPDH in whole cell lysates (10 μg protein/lane).

FIG. 17 shows hUC-MSC sheets prepared in culture medium containing human platelet lysate (hPL) (left) or fetal bovine serum (FBS) (right). The ruler shown is in cm.

FIG. 18A-18B shows immunostained fluorescence imaging of HGF expression in vivo in hUC-MSC sheets implanted within subcutaneous tissue of immuno-deficient mice. MSC sheet transplanted subcutaneous tissue sites were harvested for histological observation at 1 day (A) and 10 days (B) after implantation. The samples were stained with anti-human HGF antibody for detection of human HGF expression from hUC-MSC sheets, and cell nuclei were stained with DAPI.

FIG. 19A-19B shows hUC-MSC sheets produced with an initial cell density of 2×10⁴, 4×10⁴, 6×10⁴, 8×10⁴ or 10×10⁴ cells/cm² in the TRCD in cell culture media containing 20% FBS (A). Increasing initial cell density increased HGF gene expression in a dose-dependent manner (B).

FIG. 20A-20B shows HLA DR, DP, DQ expression in hUC-MSC single cell suspension cultures as a function of passage number (A), and in harvested MSC cell sheets (B). HLA expression was measured from passage 4 to 12 in single cell suspension cultures (A). Percentages in (A) represent the percentage of cells expressing HLA. HLA-DR gene expression was not detectable in a hUC-MSC sheet, while cell sheets prepared identically from human adipose-derived stem cells (hADSC) or human bone marrow-derived mesenchymal stem cells (hBMSC) exhibited relatively high levels of HLA-DR gene expression in comparison (B).

FIG. 21 shows a sutured uterus in a nude rat model of uterine scar development before transplantation of a hUC-MSC sheet.

FIG. 22 shows a sutured uterus in a nude rat model of uterine scar development before and after transplantation of a fluorescently labeled hUC-MSC sheet (top right image) to the rat uterus. Bright in vivo microscopy image (lower right image) indicates fluorescent cell sheet in situ on rat uterus after suturing and sheet transplantation.

FIG. 23 shows nude rat uteri harvested 1, 3, 7, or 14 days after fluorescently labeled hUC-MSC sheet transplantation. Bright images on/around uteri indicate signal from retained hUC-MSC fluorescent cell sheet.

FIG. 24 shows histological comparisons of control (no cell treatment, suture only) uterine sections versus uterine section 14 days after hUC-MSC sheet transplantation. Control uterine horn cross sections display increased fibrotic areas by dye staining compared to cell sheet uterine horn transplantation groups.

FIG. 25 compares fibrotic to myometrial areas assessed between control and hUC-MSC transplanted horns of nude rat uteri 14 days after sheet transplantation. Six rats and a total of 18 histological specimens were evaluated for fibrotic scarring areas from histological dye staining (per FIG. 24).

FIG. 26 compares fibrotic to myometrial area ratios assessed between control and hUC-MSC transplanted horns of nude rat uteri 14 days after sheet transplantation. Six rats and a total of 18 histological specimens were evaluated for fibrotic scarring areas from histological dye staining (per FIG. 24 and FIG. 25).

FIG. 27 compares thickness (microns) in scar (control) and cell sheet transplantation areas between control and hUC-MSC sheet transplanted horns of nude rat uteri 14 days after transplantation. Six rats and a total of 18 histological specimens were evaluated for fibrotic thickness from histological dye stained samples.

FIG. 28A-28B shows a nude rat uterine scar model and hUC-MSC cell sheet transplantation procedure after sheet fluorescent dye staining. (a) Schematic drawing (see i-vi) of the cell sheet transplantation process: i) surgically exposing uterine horns, ii) opening both uterine cavities, iii) closing the wounds, iv) harvested dye-labeled human stem cell sheet cartoon with corresponding actual bright field sheet micrograph image below, v) cartoon of cell sheet stained by green fluorescent dye with corresponding actual fluorescent sheet micrograph image below, vi) transplantation of labeled cell sheet onto the rat left uterine horn only (right horn control). (b) Gross in vivo photographs of hUC-MSC cell sheet transplantation process in situ in a nude rat. ii) (corresponding to element ii in the schematic drawing) surgically opened uterine cavity with dashed line indicating endometrial surface, iii) uterine wound closure, with black arrows indicating sutured uterine site, vi) transplantation of fluorescently stained hUC-MSC sheet, with white arrows indicating transplanted cell sheet on uterine suture line.

FIG. 29A-29C shows hUC-MSC cell sheet fabrication. (a) Top-down microscopic image morphology of hUC-MSCs in culture. Left column: Day 1 of seeding; Right column: Day 5 after seeding, cells are confluent on culture surface. Scale bar=200 μm in upper microscopy images and 500 μm in lower microscopy images, (b) Gross observation of harvested cell sheet. After reducing culture temperature to 20° C., cell sheets are harvested spontaneously and naturally. Scale=1 mm (c) Histological stained cross-sectional image of released hUC-MSC cell sheet. Scale bar=200 μm (left) and 50 μm (right); right micrograph indicates expanded inset box from left image to show individual stem cell nuclei (dark) and connected cell bodies with expressed cell-cell junctions and extracellular matrix.

FIG. 30A-30B shows tracking of a fluorescently stained hUC-MSC sheet. (a) Nude rat uterine gross observation imaged using in situ fluorescent microscopy and bright field microscopy in top and second row, respectively. Horizontal cross-sectional fluorescent image of uterine horn in third row. Histological imaging of cell sheet fluorescence in the bottom row. Scale bar=100 μm Bright fluorescence signal (arrows) from cell sheet confirmed in situ at 1 and 3 days after rodent horn transplantation. A small fluorescently stained fragment (arrow) was confirmed at 7 days post-transplantation. No stained cells are observed at 14 days post-transplantation. (b) Gene expression of human hepatocyte growth factor (HGF) and human vascular endothelial growth factor (VEGF) from cell sheet-transplanted uteruses at 1, 3, 7 and 14 days after transplantation. Gene expression of human HGF message and human VEGF message from scarred control uterus (no cell sheet) was not detected at all days. Human gene expression differences between the cell sheet transplantation and control groups are significant at days 1 and 3 (*p<0.01). Scarred control uterine gene expression was not detectable, and data are not shown.

FIG. 31A-31E shows assessment of fibroblast numbers at 3 days post-transplantation in rat uterine horn tissue samples (a-d) Cross-sectional HE-stained histological microscopy images (a, b), and immunohistology-stained S100A4 protein images, specific for fibroblasts (c, d), from rat uterine horns. Scale bar=500 μm. Dark dashed lines in histological images indicate rat uterine myometrium layer. Black arrows indicate transplanted human umbilical cord mesenchymal stem cell sheet. S100A4-positive stained cells (fibroblasts) in uterine scar control group show higher abundance than in cell sheet transplantation group. (e) Graph comparing S100A4-positive cell numbers in rat uterine scar control and hUC-MSC sheet uterine transplantation groups. S100A4-positive cells in scar control group are significantly higher than that in hUC-MSC sheet transplantation group (*p<0.05, n=18).

### DETAILED DESCRIPTION OF THE INVENTION

This disclosure describes decreased uterine scar formation and increased myometrial regeneration resulting from transplantation of a mesenchymal stem cell (MSC) sheet. For example, this disclosure describes decreased formation of fibrotic tissue and increased myometrial regeneration following transplantation of a human umbilical cord mesenchymal stem cell (hUC-MSC) sheet to a sutured incision site of a uterus, indicating that applying the hUC-MSC sheet reduced uterine scar formation. In the control group, a large fibrotic area was present between the host myometrium areas as a result of wound healing. In contrast, the fibrotic area in the hUC-MSC sheet transplantation group was significantly smaller than in the control group. Specifically, application of the hUC-MSC sheet to the uterus reduced the fibrotic surface area of the uterus by approximately 27% relative to the control group, and reduced the ratio of fibrotic-to-normal myometrial surface by greater than 33% relative to the control group. Thus transplantation of the hUC-MSC sheet significantly reduced fibrosis on the myometrial surface. Accordingly, the hUC-MSC sheets described herein improve healing of the uterine scar and have the potential to decrease morbidities related to abnormal uterine scar formation.

This disclosure also describes methods of preparing MSC sheets for use in reducing uterine scar formation. For example, hUC-MSCs were used to prepare cell sheets in vitro using temperature-responsive cell culture dishes (TRCDs) coated with a temperature-responsive polymer. Confluent cell sheets formed at 4-6 days after seeding and were detached from the TRCD by cooling the cultures to room temperature. Various culture conditions were identified that allow for successful production of robust, uniform hUC-MSC sheets containing one or more layers of aggregated, confluent cells. These culture conditions included optimization of subculture (passage) number before adding cells to the TRCD, initial cell density in the TRCD, addition of cell growth factors such as human platelet lysate (hPL) to the cell culture solution, and culture time in the TRCD before detachment from the temperature-responsive polymer.

I. Mesenchymal Stem Cells (MSCs)

Mesenchymal stem cells (MSCs) suitable for use in the methods described herein include, but are not limited to MSCs from umbilical cord, cord blood, limb bud, bone marrow, dental tissue (e.g. molars), adipose tissue, muscle and amniotic fluid.

In a particular embodiment, the mesenchymal stem cell is a human umbilical cord mesenchymal stem cell. The term “human umbilical cord mesenchymal stem cell” or “hUC-MSC” as used herein refers to a mesenchymal stem cell that has been isolated from a human umbilical cord.

Mesenchymal stem cells (MSCs) have a remarkable clinical potential to treat a wide range of debilitating diseases, mainly due to their unique immunomodulatory role and regenerative capacity (Caplan and Sorrell, 2015, Immunol Lett 168(2): 136-139). A convenient source for human MSCs is the umbilical cord, which is discarded after birth and provides an easily accessible and non-controversial source of stem cells for therapy (El Omar et al., 2014, Tissue Eng Part B Rev 20(5): 523-544). hUC-MSCs have been validated for safety and efficacy in human clinical trials as suspensions (Bartolucci et al., 2017, Circ Res, 121(10), 1192-1204). Moreover, hUC-MSCs have been successfully used in experimental animal disease models (Zhang et al., 2017, Cytotherapy 19(2): 194-199).

Methods for isolating MSCs from umbilical cords are known in the art and are described, for example, in U.S. Pat. No. 9,903,176, which is incorporated by reference herein in its entirety. The human umbilical cord comprises the umbilical artery, the umbilical veins, Wharton's Jelly, and the subepithelial layer. In some embodiments, the hUC-MSCs are isolated from the subepithelial layer of the human umbilical cord. In some embodiments, the hUC-MSCs are isolated from Wharton's Jelly of the human umbilical cord. Various cellular markers may be used to identify hUC-MSCs isolated from the subepithelial layer. For example, in some embodiments, the hUC-MSCs isolated from the subepithelial layer express one or more cell markers selected from CD29, CD73, CD90, CD146, CD166, SSEA4, CD9, CD44, CD146, and CD105. In a particular embodiment, the hUC-MSCs express CD73. In some embodiments, the hUC-MSCs isolated from the subepithelial layer do not express one or more cell markers selected from CD45, CD34, CD14, CD79, CD106, CD86, CD80, CD19, CD117, Stro-1, HLA-DR, HLA-DP and HLA-DQ. In a particular embodiment, the hUC-MSCs do not express HLA-DR, HLA-DP or HLA-DQ. In some embodiments, the cell sheets described herein are prepared with mesenchymal stem cells (MSCs) with low HLA expression, e.g. less than 5%, 4%, 3%, 2%, 1%, 0.5%, 0.4%, 0.3%, 0.2%, or 0.1% of the MSCs in the cell sheet express HLA (e.g. HLA-DR, HLA-DP and/or HLA-DQ).

hUC-MSCs in the umbilical cord are surrounded by extracellular matrix (ECM) and connected with other types of umbilical cord cells (e.g. endothelial cells, epithelial cells, muscle cells, and fibroblasts) through cell-cell junction structures. In contrast to endogenous hUC-MSCs in the umbilical cord, the hUC-MSC sheets described herein comprise one or more layers of aggregated confluent hUC-MSCs in which the hUC-MSCs are connected to other hUC-MSCs, not to other types of umbilical cord cells. The hUC-MSC sheets described herein also differ from hUC-MSC suspension cultures in several ways. Suspension cultures of hUC-MSCs comprise single cells lacking an ECM or cell-cell junctions because these cell adhesive proteins in these cell-cell junctions must be removed (e.g. by proteolytic trypsin treatment) to harvest and suspend cells from culture surfaces commonly used for preparation of the cell suspension culture. In contrast to singe cell suspensions of hUC-MSCs, the hUC-MSC sheets described herein contain both an endogenous cell-produced ECM and intact cell-cell junctions among the hUC-MSCs that are generated during formation of the cell sheet. The endogenous ECM and intrinsic cell-cell junctions retained during cell sheet formation, fabrication and handling facilitate retention of important properties for their phenotypic preservation, cell functions and adhesion of the hUC-MSC sheet to target tissue during transplantation to a host organism.

II. Cell Sheets Produced from MSCs

In certain aspects the present disclosure relates to a mesenchymal stem cell sheet comprising one or more layers of confluent mesenchymal stem cells (MSCs). The term “mesenchymal stem cell sheet” or “MSC sheet” as used herein refers to a cell sheet obtained by growing mesenchymal stem cells on a cell culture support in vitro. In some embodiments, the MSCs in the MSC sheet are aggregated or physically contiguous. In some embodiments, the mesenchymal stem cell sheet is a human umbilical cord mesenchymal stem cell (hUC-MSC) sheet. The MSC sheets described herein are harvested as an intact sheet by temperature shift using a temperature-responsive culture dish (TRCD) without any enzyme treatment. The MSC sheets maintain their integrity and shape by retaining tissue-like structures, actin filaments, extracellular matrix, intercellular proteins, and high cell viability, all of which are related to improved cell survival and cellular function. Accordingly, the cell sheets described herein may comprise structural features that improve cell survival and cell function, including native extracellular matrix, cell adhesion proteins and cell junction proteins. Thus, the MSC sheets prepared by the methods described herein have several beneficial characteristics compared to MSCs produced by other methods. For example, the chemical disruption method is unable to maintain tissue-like structures of cells as well as cell-cell communication, since enzyme treatment disrupts the extracellular and intracellular proteins (cell-cell and cell-ECM junctions). Accordingly, protein cleavage by enzymes reduces cell viability and cellular functions. Physical disruption (i.e., by rubber policeman or media aspiration) produces disruption of cell-cell junctions and disintegration of the cultured adherent sheet into cell aggregates.

In some embodiments, the extracellular matrix comprises one or more proteins selected from the group consisting of fibronectin, laminin and collagen. In some embodiments, the cell junction proteins are selected from the group consisting of Vinculin, Integrin-β1, Connexin 43, β-catenin, Integrin-linked kinase and N-cadherin.

The MSCs in the cell sheets may also maintain additional structural features, such as microvilli and filopodia. Microvilli are cell membrane protrusions involved in a wide variety of cell functions, including absorption, secretion, and cellular adhesion. Filopodia are cytoplasmic projections that play a role in cell-cell interactions. Thus maintenance of these structural features may also help to maintain cell function and signaling useful for their application.

In some embodiments, the cell sheet consists of MSCs. In some embodiments, the cell sheet consists essentially of MSCs. In some embodiments, at least 50%, 60%, 70%, 80%, 90%, 95%, 96%, 97%, 98% or 99% of cells in the cell sheet are MSCs. In some embodiments, 100% of the cells in the cell sheet are MSCs.

The MSCs may be added to the culture solution on the temperature-responsive polymer in the cell culture support at various cell densities to optimize formation of the cell sheet or its characteristics. For example, cytokine expression levels in the MSC may be optimized by controlling the initial cell density of the MSCs in the cell culture support (e.g. TRCD). In some embodiments, increasing the initial cell density of the MSCs in the cell culture support increases cytokine expression (e.g. HGF). In some embodiments, increasing the initial cell density of the MSCs in the cell culture support decreases cytokine expression. In some embodiments the initial cell density of the MSCs in the cell culture support used for preparation of the cell sheet is from 0.5×10⁴/cm² to 9×10⁵/cm². In some embodiments, the initial cell density of the MSCs in the cell culture support is at least 0.5×10⁴, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, or 9×10⁵ cells/cm². Any of these values may be used to define a range for the initial cell density of the MSCs in the cell culture support. For example, in some embodiments, the initial cell density in the cell culture support is from 2×10⁴ to 1×10⁵ cells/cm², 4×10⁴ to 1×10⁵ cells/cm², or 1×10⁴ to 5×10⁴ cells/cm².

The MSC sheets described herein may be transplanted to a target tissue in a host organism (e.g. a human) for therapeutic uses. Transplantation of the MSC sheets to the target tissue may prompt formation of new blood capillaries (angiogenesis) in the host tissue, as well as blood vessel formation between the transplanted cell sheet and the host tissue. This neocapillary formation is an important capability for engraftment, viability and tissue regeneration. In addition, this new blood vessel recruitment into sheets on the target tissue suggests that implanted MSC sheets continually secret paracrine factors to modulate this engraftment.

In some embodiments, the MSC sheets express one or more cytokines, for example, one or more anti-inflammatory cytokines and/or one or more inflammatory cytokines. In some embodiments the anti-inflammatory cytokine is derived from hepatocyte growth factor (HGF), fibroblast growth factor (FGF), vascular endothelial growth factor (VEGF) and interleukin-10 (IL-10). In some embodiments, the inflammatory cytokine is tumor necrosis factor-α (TNF-α). In some embodiments, cytokine expression (e.g. an anti-inflammatory cytokine or an inflammatory cytokine) in the cell sheet is increased relative to a suspension of MSCs containing an equivalent number of cells. In some embodiments, expression of the cytokine (e.g. an anti-inflammatory cytokine or an inflammatory cytokine) is decreased relative to a suspension of MSCs containing an equivalent number of cells. For some therapeutic uses, reducing secretion of inflammatory cytokines by the cell sheet would be beneficial. For example, in a particular embodiment, the cell sheet secretes tumor necrosis factor-α (TNF-α) into a culture solution in vitro at a rate of less than 100, 90, 80, 70, 60, 50, 40 or 30 pg/mL of culture solution/24 hours.

The MSC sheets described herein may continue to express cytokines after transplantation to a target tissue in a host organism. In some embodiments, the cell sheet expresses the cytokine for at least 1, 2, 3, 4, 5, 6, 7, 8, 9, 10, 15, 20, 25 or 30 days after transplantation to a tissue in a host organism. In some embodiments, the cell sheet expresses the cytokine for at least 1, 2, 3, 4, 5 or 6 months after transplantation to a tissue in a host organism.

The MSC sheets described herein may also continue to express extracellular matrix proteins and cell junction proteins after transplantation to a target tissue in a host organism. For example, in some embodiments the cell sheet expresses extracellular matrix proteins and/or cell junction proteins for at least 1, 2, 3, 4, 5, 6, 7, 8, 9, 10, 15, 20, 25 or 30 days after transplantation to a tissue in a host organism. In some embodiments, the cell sheet expresses the extracellular matrix proteins and/or cell junction proteins for at least 1, 2, 3, 4, 5 or 6 months after transplantation to a tissue of a host organism. In some embodiments the extracellular matrix proteins expressed in the cell sheet after transplantation are selected from fibronectin, laminin and collagen. In some embodiments the cell junction proteins expressed in the cell sheet after transplantation are selected from Vinculin, Integrin-β1, Connexin 43, β-catenin, Integrin-linked kinase and N-cadherin.

Current stem cell therapies often use cultured stem cells isolated from biopsies as injectable cell suspensions (Bayoussef et al., 2012, J Tissue Eng Regen Med, 6(10)). Injected cell suspensions typically exhibit lower engraftment into and retention within diseased organs or tissues (Devine et al., 2003, Blood, 101(8), 2999-3001). Loss of intact ECM and cell-cell junctions (i.e., communication) in stem cell suspensions through enzymatic disruption at harvest compromises stem cell function, engraftment and survival in vivo, and can limit therapeutic efficacy in vivo. In contrast, the methods of preparing MSC sheets described herein preserve intrinsic cell functional structures, improving attachment of the cell sheet to the target tissue after transplantation. For example, in some embodiments, the cell sheet remains attached to the target tissue in the host organism for at least 1, 2, 3, 4, 5, 6, 7, 8, 9, 10, 15, 20, 25 or 30 days after transplantation to a tissue in a host organism. In some embodiments, the cell sheet remains attached to the target tissue in the host organism for at least 1, 2, 3, 4, 5 or 6 months after transplantation to a tissue of a host organism.

Human leukocyte antigens (HLAs) are cell-surface proteins that make up the major histocompatibility complex (MHC) proteins in humans and are responsible for regulation of the immune system. HLA markers are important to control for tissue transplantation and host acceptance. HLAs corresponding to MHC class II (DP, DM, DO, DQ, and DR) present antigens from the cell surface to host T-lymphocytes to modulate host recognition as “self”. These antigens stimulate the multiplication of T-helper cells (CD4⁺ T cells), which in turn stimulate antibody-producing B-cells to produce antibodies to that specific antigen. Thus minimizing expression of HLAs is beneficial for minimizing a host immune response to transplanted hUC-MSC sheets in a host organism. In some embodiments, the hUC-MSC sheets described herein do not express one or more of Human Leukocyte Antigen-DR isotype (HLA-DR), Human Leukocyte Antigen-DP isotype (HLA-DP), or Human Leukocyte Antigen-DQ isotype (HLA-DQ). In some embodiments, less than 5%, 4%, 3%, 2%, 1%, 0.5%, 0.4%, 0.3%, 0.2%, or 0.1% of the hUC-MSCs in the cell sheet express HLA (e.g. HLA-DR, HLA-DP and/or HLA-DQ).

III. Methods for Producing MSC Sheets In Vitro

In certain aspects, the present disclosure relates to a method for producing a cell sheet comprising one or more layers of aggregated confluent mesenchymal stem cells (MSCs), the method comprising:
a) culturing MSCs in culture solution on a temperature-responsive polymer surface, for example, as frequently found coated as a thin film onto a substrate surface of a cell culture support, wherein the temperature-responsive polymer has a lower critical solution temperature in water of 0-80° C.;
b) adjusting the temperature of the culture solution to below the polymer's lower critical solution temperature, whereby the substrate surface is made hydrophilic and adhesion of the cell sheet to the surface is weakened; and
c) detaching the cell sheet from the culture support.

In some embodiments, the MSCs are human umbilical cord mesenchymal stem cells (hUC-MSCs). Methods for isolating hUC-MSCs are known in the art and are described, for example, in U.S. Pat. No. 9,803,176, which is incorporated by reference herein in its entirety. For example, hUC-MSCs may be isolated from the subepithelial layer of an umbilical by washing the umbilical cord to remove blood, Wharton's Jelly, and any other material, and dissecting the subepithelial layer (SL) from the umbilical cord. The cord tissue may be washed multiple times in a solution of Phosphate-Buffered Saline (PBS) such as Dulbecco's Phosphate-Buffered Saline (DPBS). The PBS can include a platelet lysate (i.e. 10% PRP lysate of platelet lysate). The SL can then be placed interior side down on a substrate. An entire dissected umbilical cord with the Wharton's Jelly removed can be placed directly onto the substrate, or the dissected umbilical cord can be cut into smaller sections (e.g. 1-3 mm) and these sections can be placed directly onto the substrate. The substrate can be a solid polymeric material such as a cell culture dish. The SL can be placed upon the substrate of the cell culture dish without any additional pretreatment to the cell culture treated plastic, or on a semi-solid culture medium such as agar. Following placement of the SL on the substrate, the SL is cultured in a suitable medium (e.g. Dulbecco's Modified Eagle Medium (DMEM) glucose (500-6000 mg/mL) without phenol red, 1× glutamine, 1×NEAA, and 0.1-20% PRP lysate or platelet lysate). The culture can then be cultured under either normoxic or hypoxic culture conditions for a period of time sufficient to establish primary cell cultures (e.g. 3-7 days). After primary cell cultures have been established, the SL tissue is removed and discarded. Cells or stem cells are further cultured and expanded in larger culture flasks in either a normoxic or hypoxic culture conditions.

General methods for preparing cell sheets are known in the art and are described, for example, in U.S. Pat. Nos. 8,642,338; 8,889,417; 9,981,064; and 9,114,192, each of which is incorporated by reference herein in its entirety.

The temperature-responsive polymer used to coat the substrate of the cell culture support has an upper or lower critical solution temperature in aqueous solution which is generally in the range of 0° C. to 80° C., for example, 10° C. to 50° C., 0° C. to 50° C., or 20° C. to 45° C.

The temperature-responsive polymer may be a homopolymer or a copolymer. Exemplary polymers are described, for example, in Japanese Patent Laid-Open No. 211865/1990. Specifically, they may be obtained by homo- or co-polymerization of monomers such as, for example, (meth)acrylamide compounds ((meth)acrylamide refers to both acrylamide and methacrylamide), N-(or N,N-di)alkyl-substituted (meth)acrylamide derivatives, and vinyl ether derivatives. In the case of copolymers, any two or more monomers, such as the monomers described above, may be employed. Further, those monomers may be copolymerized with other monomers, one polymer may be grafted to another, two polymers may be copolymerized, or a mixture of polymer and copolymer may be employed. If desired, polymers may be crosslinked to an extent that will not impair their inherent properties.

The substrate which is coated with the polymer may be of any types including those which are commonly used in cell culture, such as glass, modified glass, silicon oxide, polystyrene, poly(methyl methacrylate), polyester, polycarbonate, and ceramics.

Methods of coating the support with the temperature-responsive polymer are known in the art and are described, for example, in Japanese Patent Laid-Open No. 211865/1990. Specifically, such coating can be achieved by subjecting the substrate and the above-mentioned monomer or polymer to, for example, electron beam (EB) exposure, irradiation with γ-rays, irradiation with UV rays, plasma treatment, corona treatment, or organic polymerization reaction.

The coverage of the temperature responsive polymer may be in the range of 0.4-3.0m/cm², for example, 0.7-2.8 μg/cm², or 0.9-2.5 μg/cm². The morphology of the cell culture support may be, for example, a dish, a multi-plate, a flask or a cell insert.

The cultured cells may be detached and recovered from the cell culture support by adjusting the temperature of the support material to the temperature at which the polymer on the support substrate hydrates, whereupon the cells can be detached. Smooth detachment can be realized by applying a water stream to the gap between the cell sheet and the support. Detachment of the cell sheet may be affected within the culture solution in which the cells have been cultivated or in other isotonic fluids, whichever is suitable.

In a particular embodiment, the temperature-responsive polymer is poly(N-isopropyl acrylamide) Poly(N-isopropyl acrylamide) has a lower critical solution temperature in water of 31° C. If it is in a free state, it undergoes dehydration in water at temperatures above 31° C. and the polymer chains aggregate to cause turbidity. Conversely, at temperatures of 31° C. and below, the polymer chains hydrate to become dissolved in water, thereby causing release of the cell sheet from the polymer. In a particular embodiment, this polymer covers the surface of a substrate such as a Petri dish and is immobilized on it. Therefore, at temperatures above 31° C., the polymer on the substrate surface also dehydrates but since the polymer chains cover the substrate surface and are immobilized on it, the substrate surface becomes hydrophobic. Conversely, at temperatures of 31° C. and below, the polymer on the substrate surface hydrates but since the polymer chains cover the substrate surface and are immobilized on it, the substrate surface becomes hydrophilic. The hydrophobic surface is an appropriate surface for the adhesion and growth of cells, whereas the hydrophilic surface inhibits the adhesion of cells and the cells are detached simply by cooling the culture solution.

Culture solutions for mesenchymal stem cells are known in the art and are described, for example, in U.S. Pat. Nos. 9,803,176 and 9,782,439, each of which is incorporated by reference herein in its entirety. In some embodiments, the cell culture solution comprises human platelet lysate (hPL). In some embodiments, the culture solution comprises fetal bovine serum (FBS). In some embodiments, the culture solution comprises ascorbic acid. In some embodiments, the culture solution is a xeno-free medium, i.e. a medium that may contain products obtained from humans but does not contain products obtained from non-human animals. In some embodiments, the culture solution contains at least one product obtained from a non-human animal (e.g. FBS). In some embodiments, the culture solution does not contain a product obtained from a human.

In a particular embodiment, the culture solution comprises one or more of Dulbecco's Modified Eagle's Medium (DMEM) (Life Technologies, CA, USA), human platelet lysate (hPL, iBiologics, Phoenix, USA), Glutamax (Life Technologies), MEM Non-Essential Amino Acids Solution (NEAA) (Life Technologies) and an antibiotic, e.g. penicillin streptomycin.

The MSCs (e.g. hUC-MSCs) may be passed through one or more subcultures (i.e. passages) prior to culturing the cells in culture solution on a temperature-responsive polymer which has been coated onto a substrate surface of a cell culture support. In some embodiments, the MSCs (e.g. hUC-MSCs) are passed through 1, 2, 3, 4, 5, 6, 7, 8, 9, 10, 11, 12, 13, 14 or 15 subcultures prior to culturing the cells in culture solution on a temperature-responsive polymer which has been coated onto a substrate surface of a cell culture support. Any of these values may be used to define a range for the number of subcultures. For example, in some embodiments, the MSCs (e.g. hUC-MSCs) are passed through 2 to 10, 4 to 8, or 1 to 12 subcultures prior to culturing the cells on a temperature-responsive polymer. In some embodiments the number of subcultures is less than 1, 2, 3, 4, 5, 6, 7, 8, 9, 10, 11, 12, 13, 14 or 15. In some embodiments, the number of subcultures is at least 1, 2, 3, 4, 5, 6, 7, 8, 9, 10, 11, 12, 13, 14 or 15.

The MSC sheet may be prepared in a range of different sizes depending on the application. In some embodiments, the MSC sheet has a diameter of at least 1, 2, 3, 4, 5, 6, 7, 8, 9, 10, 15 or 20 cm. Any of these values may be used to define a range for the size of the MSC sheet. For example, in some embodiments, the MSC sheet has a diameter from 1 to 20 cm, from 1 to 10 cm or from 2 to 10 cm. In some embodiments, the MSC sheet has an area of at least 1, 2, 3, 4, 5, 6, 7, 8, 9, 10, 15, 20, 30, 40, 50, 60, 70, 80, 90, 100, 150, 200, 250 or 300 cm². Any of these values may be used to define a range for the size of the MSC sheet. For example, in some embodiments, the MSC sheet has an area from 1 to 100 cm², 3 to 70 cm², or 1 to 300 cm². The methods described herein result in an hUC-MSC sheet in which the surface area of the hUC-MSC sheet is much greater than its thickness. For example, in some embodiments the ratio of the surface area of the hUC-MSC sheet to its thickness is at least 10:1, 100:1, 1000:1, or 10,000:1. The hUC-MSC sheets described herein comprise one or more layers of confluent human umbilical cord mesenchymal stem cells (hUC-MSCs), for example, 1, 2, 3, 4, 5, 6, 7, 8, 9 or 10 layers of hUC-MSCs. In some embodiments, the hUC-MSC sheet comprises fewer than 2, 3, 4, 5, 6, 7, 8, 9 or 10 layers of hUC-MSCs. In some embodiments, the hUC-MSC sheet comprises at least 1, 2, 3, 4, 5, 6, 7, 8, 9 or 10 layers of hUC-MSCs.

IV. Methods for Using MSC Sheets to Reduce Formation of Fibrotic Tissue and Increase Myometrial Regeneration in the Uterus

In some aspects, the present disclosure relates to a method of reducing formation of fibrotic tissue in a uterus of a subject in need thereof, comprising applying a mesenchymal stem cell (MSC) sheet to the uterus of the subject, wherein the SC sheet comprises one or more layers of aggregated confluent mesenchymal stem cells (MSCs), and wherein applying the −MSC sheet to the uterus reduces the formation of fibrotic tissue in the uterus relative to a uterus in which the MSC sheet is not applied.

In some aspects, the present disclosure relates to a method of increasing myometrial regeneration in a uterus of a subject in need thereof, comprising applying a mesenchymal stem cell (MSC) sheet to the uterus of the subject, wherein the MSC sheet comprises one or more layers of aggregated confluent mesenchymal stem cells (MSCs), and wherein applying the MSC sheet to the uterus increases myometrial regeneration relative to a uterus in which the MSC sheet is not applied.

The uterus comprises four layers, the endometrium epithelium, endometrium stroma, myometrium and perimetrium. The endometrium comprises epithelial and stromal layers as inner layers. It has a basal layer and a functional layer; the functional layer thickens and then is sloughed during the menstrual cycle or estrous cycle. The myometrium is the middle layer of the uterine wall, consisting mainly of uterine smooth muscle cells (also called uterine myocytes), but also of supporting stromal and vascular tissue. The main function of the myometrium is to induce uterine contractions. The outer layer of the uterus is the perimetrium.

For Caesarean delivery, an incision of about 15 cm is typically made through the mother's lower abdomen and the uterus is then opened with a second incision and the baby delivered. The incisions are then stitched closed in multiple tissue layers.

For other uterine surgeries such as myomectomy which is removed myoma nodules, an incision of any length and location depended on size of lesions are made through the patient's lower abdomen or abdominal hole for endoscopic surgeries and the lesions are removed with an incision in border line between normal tissue and abnormal tissue. The incisions are then stitched closed in single or multiple tissue layers. After uterine surgeries, the initial scar is typically fibrous tissue; and that fibrotic scar is weak, prone to rupture and other problems, and in need of mitigation with normal scar formation, remodeling and myometrial regeneration.

MSC sheets may also be used after non-surgical procedures. For example, in some embodiments, the MSC sheet is applied to the uterus after dilation and curettage (D&C) (e.g. after miscarriage) or after removal of uterine fibroids. The MSC sheets could be applied with an endoscope deploying cell sheets vaginally.

One advantage of the MSC sheets described herein is that the extracellular matrix of the applied cell sheet acts as a natural adhesive to bind the cell sheet to the uterine tissue of the subject, such that suturing or stitching is not required to adhere the cell sheet to the tissue. A support membrane or other devices may be used to transfer the MSC sheet to the uterine tissue of the subject and then removed after sheet transfer. The supports can be, for example, poly(vinylidene difluoride) (PVDF), cellulose acetate, cellulose esters, plastic and metal. The MSC sheets readily adhere to target tissue, self-stabilizing without suturing after being placed directly onto the target tissue for a short period of time. For example, in some embodiments, the MSC sheet adheres to the target tissue within 5, 10, 15, 20, 25, or 30 minutes after contact with the tissue. Once the MSC sheet has adhered to the uterine tissue, the support membrane may be excised. In certain embodiments, the MSCs in the cell sheet are allogeneic to the subject, i.e. are isolated from a different individual from the same species as the subject, such that the genes at one or more loci are not identical. In certain reported cases, MSCs seemingly avoid allogeneic rejection in humans and in animal models (Jiang et al., 2005, Blood, 105(10), 4120-4126). Thus, the MSC sheets described herein may be used in allogeneic cell therapies as an off-the-shelf product.

Allogeneic cell sources must be capable of eliciting meaningful therapies under standard immunologic competence in host patient allogeneic tissues. This includes reliable cell homing to and fractional dose engraftment or retention for sufficient duration at the tissue site of therapeutic interest (Leor et al., 2000, Circulation, 102(19 Suppl 3), III 56-61). Current estimates are that when stem cell suspensions are administered to a subject, less than 3% of injected stem cells are retained in damaged myocardium 3 days post-injection following ischemic injury (Devine et al., 2003, Blood, 101(8), 2999-3001). Additionally, most administered cells from a cell suspension that engraft into target tissue will die within the first few weeks (Reinecke & Murry, 2002, J Mol Cell Cardiol, 34(3), 251-253). In contrast, the MSC cell sheets described herein stably engraft at high fractional retention to host tissue 7 days after transplantation. Thus the MSC sheets described herein provide distinct advantages over injected or administered mesenchymal stem cell suspensions.

In some embodiments, the MSC sheet is applied to an incision site in the uterus. In some embodiments, the incision is sutured closed before the MSC sheet is applied to the uterus (e.g. to the top incision site of the myometrium). In some embodiments, applying the MSC sheet to the uterus reduces fibrotic area of the uterus by at least 5%, 10%, 15%, 20%, 25%, 30%, 40%, 50%, 60%, 70%, 80%, or 90% relative to a uterus in which the MSC sheet is not applied. In some embodiments applying the MSC sheet to the uterus reduces the thickness of the uterine scar after Caesarean Delivery by at least 5%, 10%, 15%, 20%, 25%, 30%, 40%, 50%, 60%, 70%, 80%, or 90% relative to a uterus in which the MSC sheet is not applied.

The risk of complications for Caesarean Delivery often increases if the subject has had one or move previous Caesarean Deliveries or at least one previous uterine surgery. In some embodiments, the subject has not had a previous Caesarean Delivery or any uterine surgery. In some embodiments, the subject has had at least 1, 2, 3, 4, 5, 6, 7 or 8 previous Caesarean Deliveries and at least one previous uterine surgery.

More than one MSC sheet may be applied to the uterus in the methods described herein. In some embodiments, 1, 2, 3, 4, 5, 6, 7, 8, 9, 10 or more MSC sheets may be applied to the uterus. Any of these values may be used to define a range for the number of MSC sheets applied to the uterus. For example, in some embodiments, 2-4, 3-5 or 1-10 MSC sheets are applied to the uterus.

### EXAMPLES

### Example 1. Properties of Umbilical Cord Mesenchymal Stem Cell Sheets Prepared in Xeno-Free Media

### Materials and Methods

1.1 Human Umbilical Cord Stem Cell (hUC-MSC) Culture

Banked human umbilical cord mesenchymal stem cells isolated from the subepithelial layer of human umbilical cord tissue (Jadi Cell LLC, Miami, USA IRB-35242) (Patel et al., 2013, Cell Transplant, 22(3), 513-519) were cultured in xeno-free cell culture media with Dulbecco's Modified Eagle's Medium (DMEM) (Life Technologies, CA, USA) supplemented with 10% human platelet lysate (hPL, iBiologics, Phoenix, USA), 1% Glutamax (Life Technologies), 1% MEM NEAA (Life Technologies), 1% penicillin streptomycin (Life Technologies), at 37° C. in a humidified atmosphere with 5% CO₂ for 5 days. Subculture was performed from passage 4 until passage 12. Cell culture media was changed every two days.

1.2 hUC-MSC Proliferation Rate

hUC-MSCs were seeded on 35-mm tissue culture plates (TCP) (Corning, N.Y.) at cell numbers of 5×10⁴, 1×10⁵ and 2×10⁵ cells/dish (i.e. initial cell densities of 5×10³/cm², 1×10⁴/cm², and 2×10⁴/cm², respectively) in xeno-free cell culture media. Cells on TCP were dissociated with trypsin and cell number counted using a hemocytometer at 1, 2, 3, 4, 5 and 6 days. hUC-MSCs were seeded at a cell density of 3.5×10³/cm² on 175 cm² tissue culture flasks (Corning, N.Y.) and passaged at 5 days with TrypLE (life technologies) after culturing from passage 4 until 12. Cell number was counted each passage using a hemocytometer.

1.3 hUC-MSC Characterization in Differentiation Potential

hUC-MSCs were cultured in xeno-free cell culture media for two passages on TCP. At passages 4, 6, 8, 10, and 12, cells were prepared and induced for osteogenic and adipogenic differentiation. For osteogenic differentiation, cells were plated at 5×10³ cells/cm² in 35 mm TCP dishes in xeno-free cell culture media. When 60% confluent, cells were induced with osteogenic differentiation media containing αMEM, 10 nM dexamethasone, 82 μg/mL ascorbic acid 2-phosphate, 10 mM β-glycerolphosphate (Sigma-Aldrich). Cells were cultured in osteogenic media at 37° C. for 21 days with media changed every 3 days. To detect positive differentiation, cells were fixed with cold 4% paraformaldehyde for 12 minutes and stained with Alizarin Red S-(Sigma-Aldrich) using standard protocols. For adipogenic differentiation, cells were plated at 1×10⁴ cells/cm² in 35 mm TCP dishes in xeno-free cell culture media. When 80% confluent, cells were induced with adipogenic differentiation media containing high-glucose DMEM, 100 nM dexamethasone, 0.5 mM IBMX, and 50 μM IND (all Sigma-Aldrich). Cells were cultured in adipogenic media at 37° C. for 21 days and media changed every 3 days. To detect positive differentiation, cells were fixed with cold 4% paraformaldehyde for 12 minutes and stained with Oil Red 0 (Sigma-Aldrich) using standard protocols.

1.4 hUC-MSC Surface Phenotype Assay

hUC-MSCs were cultured in xeno-free cell culture media on TCP. Cell suspensions were prepared of P6, P8, P10, and P12 HPL and FBS cultured cells. Cells were then detached enzymatically and washed once with PBS. To minimize non-specific binding of antibodies, cells were incubated with 2% w/v Bovine Serum Albumin (BSA) in PBS for 30 minutes. Cells were then aliquoted at concentrations of 3-5×10⁵/100 μL. One aliquot was reserved as an unstained control and those remaining were stained with the following antibodies: CD44, CD90 and HLA-DR, DP, DQ (Biolegend, San Diego, Calif.). Primary antibody was added to each aliquot to achieve a ratio of about 20:1 of cells in buffer to antibody. About 3-5×10⁵ cells were stained with saturating concentrations of (fluorophore)-conjugated antibodies. Cells were incubated in the dark on ice for 30 minutes. After incubation, cells were washed three times and then re-suspended in PBS. The cells were immediately analyzed by flow cytometry. Flow cytometry was performed on a Becton, Dickinson FACS Canto (BD Biosciences, Sparks, MD). Flow cytometer instruments were set using unstained cells. Cells were gated by forward versus side scatter to eliminate doublets. A minimum of 10,000 events was counted for each analysis.

1.5 hUC-MSC Sheet Preparation Using Different Initial Cell Numbers and Passage Numbers

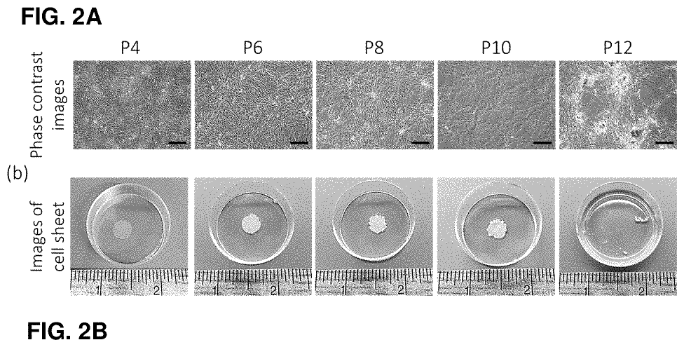

hUC-MSC sheets were prepared on temperature-responsive cell culture dishes (TRCDs) in various conditions including different initial cell density and passage numbers (FIG. 2). Passage 6 cells were seeded on 35-mm TRCDs (CellSeed Inc., Tokyo, Japan) at cell numbers of 5×10⁴ cells/dish, 1×10⁵ cells/dish and 2×10⁵ cells/dish. Passage 4-12 cells were seeded at a cell number of 2×10⁵ cells/dish (i.e. an initial cell density of 2×10⁴/cm²). Fresh xeno-free cell culture media including 16.4 μg/mL of ascorbic acid (Sigma-Aldrich, St. Louis, USA) to make cell sheets was added at 1 day after seeding. Confluent cell sheets formed at 4-6 days after seeding and were detached from TRCD at room temperature. Cell morphologies were monitored using an AX 10 microscope (Carl Zeiss Microimaging, Gottingen, Germany) with Axio Vision software (Carl Zeiss Microimaging) before cell sheets detachment.

1.6 Immunohistochemical Staining

Cultured cell sheets were removed from TRCD at room temperature and fixed with 4% paraformaldehyde for 30 min and then embedded in paraffin. Embedded specimens were sectioned into 4 μm slices and stained with H&E, stem cell surface markers, ECMs (fibronectin; FN and laminin; LM) and cell-cell junctions (integrin-linked kinase; β-catenin). For fluorescence staining (FN, LM, and β-catenin), slides were immersed in antigen retriever solution (Sigma-Aldrich) for 20 min at 100° C. and washed with PBS 1×. Non-specific binding was blocked in PBS 1× containing 10% goat serum (Vector Laboratories, Burlingame, USA), for 1 h at room temperature. Primary antibody labeling (Abcam, Cambridge, USA) (1:100) at 4° C. proceeded overnight and then washed with PBS 1×. These specimens were treated with Alexa Fluor 594-conjugated secondary antibodies (Life Technologies) (1:200) for 1 h and mounted with ProLong Gold Antifade Reagent (Life Technologies). Immunofluorescence images were obtained using an AX 10 microscope (Carl Zeiss Microimaging) and analyzed with Axiovision software (Carl Zeiss Microimaging). For H&E stain, specimens were treated with hematoxylin solution (Sigma-Aldrich) for 3 min and subsequently with eosin solution (Thermo Fisher Scientific, Kalamazoo, USA) for 5 min. The H&E stained specimens were dehydrated and mounted with Permount™ (Thermo Fisher Scientific). H&E images were obtained using a BX 41 microscope (Olympus, Hamburg, Germany).

1.7 Cell Sheet Microstructure Observed Using Transmission Electron Microscopy

hUC-MSC sheets were fixed with a mixture of 2% paraformaldehyde, 2% glutaraldehyde, 0.1 M sodium phosphate buffer, and 2% osmium tetroxide (OsO₄) in sodium phosphate buffer and dehydrated in a grade series of ethanol. Samples were then embedded in epoxy resin. Ultrathin sections (70 nm thickness) were observed with a transmission electron microscope (JEOL JEM1200EX) (JEOL USA, Peabody, USA).

1.8 Determination of Hepatocyte Growth Factor (HGF) and Tumor Necrosis Factor Alpha (TNF-α) Secretion from hUC-MSC Sheets

hUC-MSC cell sheets were fabricated on TRCDs. Supernatant media over adherent cultured cells for 24 hours was collected just prior to cell sheet detachment from TRCD at room temperature (RT). HGF and TNF-α amounts secreted from hUC-MSCs were measured by human HGF Quantikine ELISA and human TNF-α Quantikine ELISA kits, respectively (R&D Systems, Minneapolis, USA).

1.9 Cell Sheet Placement into Immune-Deficient Mice Subcutaneous Tissue

hUC-MSC (passage 6) cell sheets were detached from TRCD at RT after 4 days of culture and transplanted into subcutaneous dorsal tissues of 6-week old immune-deficient mice (NOD.CB17-Prkdc^{scid}/NCrCrl) (Charles River, San Diego, USA). Sterilized non-cytotoxic silicone membrane (Invitrogen) was placed between the cell sheet and subcutaneous dorsal tissues to prohibit tissue adhesion. Implanted mice were sacrificed 10 days after cell sheet transplantation. The cell sheet-transplanted subcutaneous tissue was fixed with 10% paraformaldehyde (Sigma-Aldrich) for 1 day for histological analysis. All procedures were approved by the Institutional Animal Care and Use Committee (IACUC) (protocol #16-12017) at The University of Utah and conducted in accordance with national guidelines.

1.10 Statistical Analyses

All quantitative values are expressed as mean and standard error (SE, mean±SE). Significant differences between groups were tested by one-way Analysis of Variance using origin 2017 software (OriginLab, Northampton, USA). A probability value of less than 0.05 (p<0.05) was considered statistically significant.

1.11 Quantitative Reverse Transcription Polymerase Chain Reaction (RT-PCR) Analysis

hUC-MSC cell sheets were collected after detachment from TRCD at RT. Total RNA from cell sheets was extracted using Trizol and PureLink RNA Mini Kit (Life Technologies) according to manufacturer's protocols. cDNA was prepared from 1 μg of total RNA using high capacity cDNA reverse transcription kits (Life Technologies). RT-PCR analysis was performed with TapMan Universal PCR Master Mix using an Applied Biosystems Step One instrument (Applied Biosystems™, Foster City, USA). Gene expression levels were assessed for the following genes: 1) glyceraldehyde 3-phosphate dehydrogenase (GAPDH, Hs02786624_g1) as a housekeeping gene, 2) integrin-linked kinase (ILK, Hs00177914_m1), 3) N-cadherin (N-cad, Hs00983056_m1). All primers were manufactured by Applied Biosystems. Relative gene expression levels were quantified by the comparative C_{T} method (Schmittgen & Livak, 2008). Gene expression levels were normalized to GAPDH expression levels. Gene expression levels are relative to the level at passage 6 cell group.

Results

hUC-MSC Sheet Preparation with Different Initial Cell Numbers and Passage Numbers

hUC-MSCs were cultured on flasks and sub-cultured using trypsin every 5 days from passages 4 to 12 (Table 1). Cells were proliferated 16-20 times from initial cell seeding numbers between passages 4-8 during sub-culture. However, cell proliferation rate dramatically decreases from passage 9. Cell numbers were 14, 10.9, 7.5, and 3.1-fold increased from initial cell seeding numbers at passage 9, 10, 11, and 12, respectively. Cells in passage 10 required one day more to reach confluence and yield cell sheets than cells in passages 4-8 at the same seeding density (FIGS. 2a and b). Cells in passage 12 exhibited heterogeneously cultured morphologies, lost contact inhibition, and clumped into multi-layered aggregates rather than consistent monolayers (FIG. 2a). When culture temperature was reduced to room temperature (RT), cells in passage 12 detached from TRCD, but not as sheets, as recovered from passages 4-10 (FIG. 2b). Cells should therefore be used from passages 4 to 8 to produce consistent cell sheet quality.

TABLE 1
Growth rates for hUC-MSCs in passages 4-12.

| | Cell | Initial cell | Expended | Fold |
|---|---|---|---|---|
| | viability | seeding density | cell density | increase |
| Passage 4 | 96% | 4 × 10³/cm² | 6.5 × 10⁴/cm² | 16.3 |
| Passage 5 | 96% | 3.5 × 10³/cm² | 6.7 × 10⁴/cm² | 19 |
| Passage 6 | 97.5% | 3.2 × 10³/cm² | 5.5 × 10⁴/cm² | 17.2 |
| Passage 7 | 96.2% | 3.2 × 10³/cm² | 5.5 × 10⁴/cm² | 17.3 |
| Passage 8 | 96.7% | 3.2 × 10³/cm² | 6.6 × 10⁴/cm² | 20.7 |
| Passage 9 | 98.5% | 3.2 × 10³/cm² | 4.5 × 10⁴/cm² | 14 |
| Passage 10 | 97.6% | 3.2 × 10³/cm² | 3.5 × 10⁴/cm² | 10.9 |
| Passage 11 | 97.4% | 3.2 × 10³/cm² | 2.4 × 10⁴/cm² | 7.5 |
| Passage 12 | 97.4% | 3.2 × 10³/cm² | 1.0 × 10⁴/cm² | 3.1 |

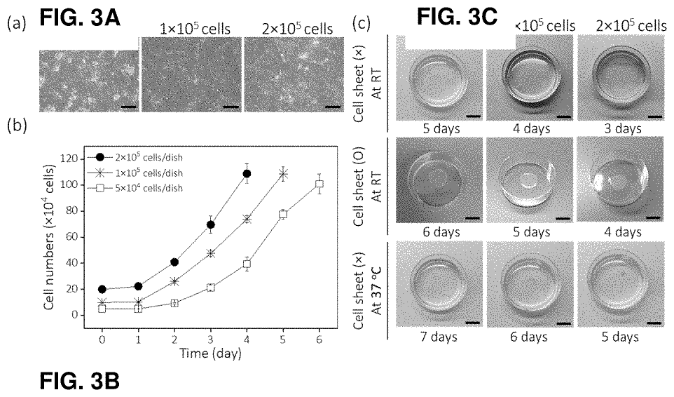

Initially seeded cell numbers of 5×10⁴, 1×10⁵, and 2×10⁵ cells/dish reached confluence at 6, 5, and 4 days, respectively (FIGS. 3a and b), reliably producing hUC-MSC sheets (FIG. 3c). Cell sheets from all different initial cell number groups spontaneously detached from TRCDs without temperature change (i.e., at 37° C.) at 5, 6, and 7 days in the 2×10⁵, 1×10⁵, and 5×10⁴ seeded cells/dish groups, respectively (FIG. 3c) once cells were over-confluent. One day prior to cell confluence, adherent cells were less than 70-80% confluent and detached as partially broken sheet fragments when temperature was reduced to RT due to insufficient cell density. Recovery of hUC-MSC sheets either one day before or one day after cells reaching confluence on TRCDs was not possible (FIG. 3c). These results indicate that cell sheets must be prepared carefully and recovered at the precise time-point when cells are neither under- or over-confluent in order to detach the cells as a thermally recovered sheet.

hUC-MSC Surface Marker Characterization

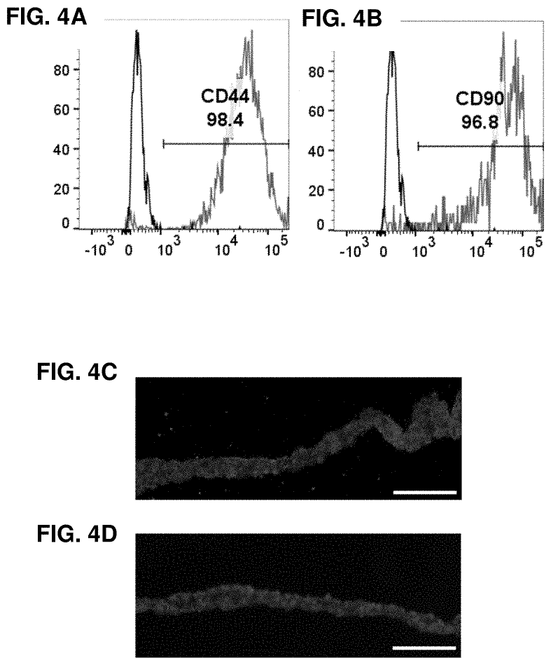

CD44 and CD90 expression was measured in hUC-MSC suspension cultures and in hUC-MSC sheets in vitro. As shown in FIG. 4, hUC-MSCs expressed CD44 and CD90 in suspension cultures (FIGS. 4A and 4B) and in cell sheets (FIGS. 4C and 4D) in vitro. CD44 and CD90 are known to be expressed in hUC-MSCs. Accordingly, these results indicate that hUC-MSC sheets maintained hUC-MSC specific phenotypes. In particular, the results in FIGS. 4C and 4D indicate that the cell sheets contain hUC-MSCs and do not contain differentiated cells and other cell types from the umbilical cord.

Structural Analysis of hUC-MSC Sheets

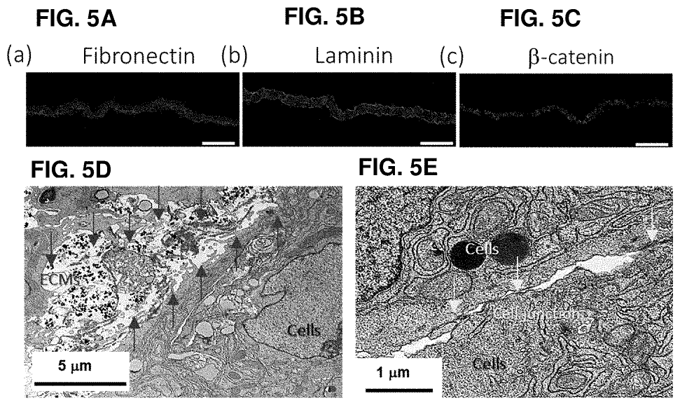

Passage 6 cells were cultured on TRCD for 4 days and resulting cell sheets were recovered from TRCD with temperature reduction to RT. The cell sheet was stained with fibronectin, laminin, and β-catenin to verify hUC-MSC sheet retention of functional structures during culture and after sheet detachment. Fibronectin and laminin, important ECM components that promote cell and tissue attachment (Yue, 2014, J Glaucoma 23: S20-S23; Kim et al., 2016, Int Neurourol J.: S23-S29), were strongly expressed across the entire cell sheet surface (FIGS. 5a and b). β-catenin, part of the protein complex forming cell adherent junctions (Nelson & Nusse, 2004, Science, 303(5663), 1483-1487), shows prominent staining between cells (FIG. 5c). Retention of ECM and cell junction proteins indicates that functional proteins produced during culture are preserved after cell sheet harvest.

Inter-cellular structures within cell sheets were observed by TEM. Horizontal sectioning showed ECM structures within cell sheets (FIG. 5d), including numerous cell-cell junctions (FIG. 5e). These results suggest that hUC-MSC sheets structurally retain functional proteins related to natural cell functions such as cell communication and cell adhesion.

Secretion of Hepatocyte Growth Factor (HGF) and Tumor Necrosis Factor-Alpha (TNF-α)

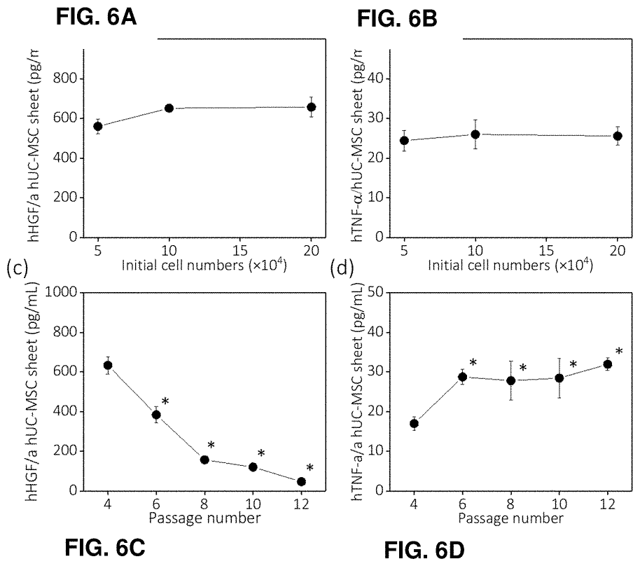

Human anti-inflammatory cytokine HGF (Gong, Rifai, & Dworkin, 2006; J Am Soc Nephrol, 17(9), 2464-2473), and pro-inflammatory cytokine TNF-α (Ertel et al., 1995, J Cell Sci, 123(Pt 24), 4195-4200) (REF) secreted from hUC-MSCs in culture supernatant were measured to support paracrine effects of the fabricated hUC-MSC sheets in vitro. No significant differences in amounts of hHGF were seen in 2×10⁵, 1×10⁵, and 5×10⁴ cells/dish groups at passage 6 (FIG. 6a). Pro-inflammatory cytokine (hTNF-α) was barely detectable in 2×10⁵, 1×10⁵, and 5×10⁴ cells/dish groups (FIG. 6b).

hUC-MSC sheets fabricated using passage 4 cells secreted significantly higher concentrations of hHGF (633 pg/mL), compared to hUC-MSC sheets fabricated using passage 6, 8, 10, and 12 cells. Amounts of hHGF secreted from hUC-MSC sheets dramatically decreased as passage number increased (FIG. 6c). hTNF-α was barely secreted (16-35 pg/mL) from hUC-MSC sheets (FIG. 6d) and hUC-MSC sheets fabricated using passage 4 had significantly lower concentrations of hTNF-α, compared to hUC-MSC sheets fabricated using passage 6, 8, 10, and 12 cells. Results therefore demonstrate that passage number is an important factor in hUC-MSC sheet cytokine properties.

Cell Sheet Implantation into Immune-Deficient Mice

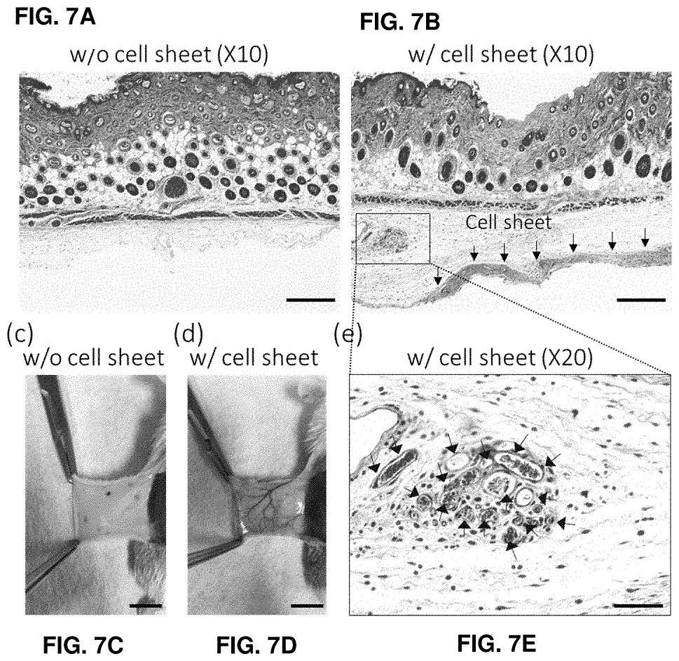

hUC-MSC sheets were implanted into dorsal subcutaneous pockets in immune-deficient mice for 10 days to demonstrate stability and engraftment in vivo. At 10 days after transplantation, formation of capillaries (angiogenesis) was observed in cell sheet-transplanted tissue, while subcutaneous tissue without cell sheet transplantation showed only a few fine blood vessels (FIGS. 7c and d). H&E staining data demonstrated that cell sheets remained localized on the transplanted area for 10 days after transplantation (FIGS. 7a and b). In cell sheet-transplanted groups, a large number of blood vessel structures was observed between transplanted cell sheets and host tissue (FIG. 7e). This indicates that the cell sheets are transplantable, engraft and preserve cell sheet structures for 10 days in vivo. Furthermore, cell sheets induce neocapillary formation as an important capability for engraftment, viability and tissue regeneration.

Discussion

Xeno-free hUC-MSC sheet fabrication was demonstrated from cultures using temperature responsive culture dishes (TRCD). These hUC-MSC sheets exhibit: 1) retention of native functional inter-cellular structures essential to cell-cell communication, act as a natural matrix adhesive when implanted onto target organs (FIG. 5); 2) hepatocyte growth factor (HGF) secretion inducing angiogenesis and anti-fibrotic action (FIG. 6); 3) cell retention in vivo for 10 days after implantation; and 4) vascular neogenesis in vivo supporting sheet-tissue engraftment (FIG. 7).

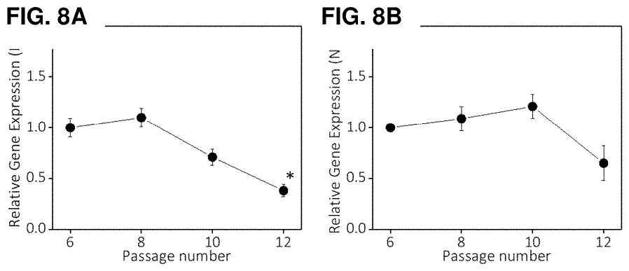

To fabricate potent MSC cell sheets reproducibly, hUC-MSCs from passages 4 to 12 were expanded and transformed to sheets in cell culture media supplemented with hPL. Cell proliferation rates for hUC-MSCs were remarkably reduced after passage 10, affecting the cell sheet creation process and timelines to harvest (FIG. 2). Furthermore, passage 12 cells were not able to form stable sheets due to reduced cell proliferation rates and inadequate cell-cell junction formation after increased passaging (Table 1 and FIG. 8). In addition, microscopy phase contrast images (FIG. 2) showed cells stacked on top of each other and formation of cell aggregates at higher passage numbers. This feature tends to increase as passage number increases, especially for passage 12 cells. Cell aggregations using bone marrow derived (BMSCs) and adipose derived (ADSC) stem cells is reported to occur when cultured in media including 5% hPL (Hemeda, Giebel, & Wagner, 2014, Cytotherapy, 16(2), 170-180). Active coagulation factors in hPL could be involved in inducing such aggregation. This cell aggregations interrupts homogeneous cell growth and cell sheet fabrication. To prepare reproducible hUC-MSC sheets, passage numbers below passage 10 would be preferable.

Rapid growth of hUC-MSCs cultured in cell culture media including hPL could be beneficial in reducing time required to fabricate cell sheets. Conversely, it may also introduce some processing difficulties because sheet cultures reach confluence quickly and are prone to spontaneous detachment upon reaching confluence. Therefore, judicious use of appropriate initial cell seeding numbers is important for the hUC-MSC sheet fabrication process. Initial cell seeding density higher than 2×10⁵ cells/dish does not yield a monolayer sheet: such high density induces spontaneous cell detachment from TRCDs within 2 days of cell culture (data not shown). In this study, initial densities of 2×10⁵, 1×10⁵, and 5×10⁴ cells were used and all successfully yielded hUC-MSC sheets at day 4, 5, and 6, respectively, when each culture reached confluence (FIG. 3). Poor cell sheet quality was observed when cells were detached one day prior to or one day after reaching confluence (FIG. 3c). Therefore, for best hUC-MSC sheet fabrication, the harvest time for TRCD cell sheet recovery depends on both passage number and seeding cell density.

Central to these results is the reliable capability to produce a stable, robust layer of hUC-MSCs using a commercial TRCD grafted with temperature-responsive polymer coating that facilitates cell harvest without destructive enzymes using temperature reduction (Okano et al., 1995, Biomaterials, 16(4), 297-303; Okano et al., 1993, J Biomed Mater Res, 27(10), 1243-1251). This cell sheet technology produces cultured cell recovery with intact native cell-cell organization, cell-cell communication, intact ECMs, and tissue-like phenotypes. Cell sheets recovered from TRCDs by small changes in culture temperature preserve cell surface-associated ECMs such as fibronectin and laminin, and cell-cell junction proteins such as β-catenin (FIG. 5), that play important roles in promoting cell adhesion and paracrine signaling (Brownlee, 2002, Curr Opin Plant Biol, 5(5), 396-401). Cell sheets with native morphologies, confluent phenotypes and organization, cell-cell communications, intact extracellular matrix (ECM) and tissue-like behaviors can be readily transferred to target tissues (Miyahara et al., 2006, Nat Med, 12(4), 459-465. hUC-MSC sheets implanted into subcutaneous tissue sites in immune-deficient mice rapidly and spontaneously attached to subcutaneous tissue surfaces within 10 min. After 10 days in vivo, implanted cell sheets remained as intact sheets (FIG. 7).

Overall, hUC-MSC sheets display several beneficial properties for improving allogeneic MSC cell therapy. Results here have determined (1) specific conditions for reliable xeno-free hUC-MSC sheet fabrication; (2) intact features of hUC-MSC sheets that preserve important cell functional structures and paracrine effects after cell harvest from TRCDs; (3) intact hUC-MSC sheet retention in implant target tissue sites for 10 days; and (4) new blood vessel recruitment into sheets on the target tissue, suggesting that implanted hUC-MSC sheets continually secret paracrine factors to modulate engraftment.

Conclusions

hUC-MSC cell sheet technology represents a unique cellular delivery method aimed to improve MSC therapy over current injected cell suspensions. The simple fabrication method on TRCDs in hPL allows rapid xeno-free production of robust uniform hUC-MSC sheets, harvested with small changes of temperature instead of destructive proteolytic enzymes. Cell production depends on several controlled culture variables, including cell seeding density, passage number, media (hPL), and culture time and TRCDs. When cultured homogeneously under optimized conditions, hUC-MSC cell sheet reproducibility is enhanced and the hUC-MSC cell sheet production process is simplified to a routine amenable to scaling. This enables future production of hUC-MSC sheets having higher cell numbers to increase paracrine action and therapeutic benefits. Given their paracrine effects and low HLA profile, fabricated xeno-free hUC-MSC sheets represent promising tissue regeneration potential both structurally and functionally in vitro and in vivo. With reliable topical tissue site placement, high engraftment efficiency, long-term retention and survival in vivo, the hUC-MSC sheet has a potential to improve therapeutic value of allogeneic cell therapy over injected stem cells used currently.

### Example 2. Comparison of Human Umbilical Cord Mesenchymal Stem Cells (hUC-MSCs) Harvested by Temperature Change, Trypsin Treatment, and Cell Scraper

Materials and Methods

2.1 Antibodies

The following antibodies were used in this study; actin (ab8226) (Abcam, Cambridge, USA), vinculin (ab129002) (Abcam), fibronectin (ab6328) (Abcam), laminin (ab11575) (Abcam), integrin β-1 (ab179471) (Abcam), connexin 43/GJA1 (ab11370) (Abcam), YAP (#140794) (Cell Signaling Technology (CST), Massachusetts, USA), phospho-YAP (Ser127, #4911)) (CST), FAK (ab40794) (Abcam), Phospho-FAK (Tyr397, #8556) (CST), GAPDH (ab9484) (Abcam). Alexa flour 568 goat anti-rabbit, 568 goat anti-mouse, 488 goat anti-rabbit, and 488 goat anti-mouse (life technologies) were used as secondary antibodies.

2.2 Human Umbilical Cord Stem Cell (hUC-MSC) Culture

Banked human umbilical cord mesenchymal stem cells (hUC-MSCs) were isolated from the subepithelial layer of human umbilical cord tissue (Jadi Cell LLC, Miami, USA IRB-35242) and were cultured in Dulbecco's Modified Eagle's Medium (DMEM) (Gibco, Massachusetts, USA) supplemented with 10% fetal bovine serum (FBS) (Gibco), 1% GlutaMAX (Gibco), 1% MEM non-essential amino acids (NEAA) (Gibco), 100 units/mL penicillin, and 100 μg/mL streptomycin (Gibco). hUC-MSC was incubated at 37° C. with 5% CO₂ in a humidified chamber and passaged when cells reached confluent. hUC-MSC was passaged with TrypLE (Gibco) treatment for 5 minutes and subculture at 3000 cells/cm² between passages 4 and 6.

2.3 Preparation of hUC-MSC Sheet

hUC-MSCs were seeded on a 35 mm temperature responsive culture dish (TRCD) (CellSeed, Tokyo, Japan). hUC-MSC was seeded at the density of 2×10⁵ cells/dish (Day 0) and cultured to confluence (Day 5). Cell culture media including 16.4 μg/mL of ascorbic acid (Wako, Osaka, Japan) was replaced at 1 day after seeding. hUC-MSC was harvested as a mono-layer sheet from TRCD within 60 minutes by reducing the temperature to 20° C. Total cell number of hUC-MSC sheet was counted with trypan blue (Gibco) exclusion test using hemocytometer.

2.4 Hematoxylin and Eosin (H&E) Staining of hUC-MSC Sheet

Samples were fixed with 4% buffered paraformaldehyde (PFA) and embedded in paraffin. Then, the samples were cut into 4 μm-thick sections. The sections were stained with Mayer's hematoxylin and 1% eosin alcohol solution. Then, it was mounted with Permount™ (Thermo Fisher Scientific). The stained samples were visualized using a BX53 microscope (Olympus, Tokyo).

2.5 Morphological Observation of hUC-MSCs Using Scanning Electron Microscopy and Transmission Electron Microscope

For scanning electron microscopy (SEM) analysis, samples were rinsed in wash buffer (0.1M sodium cacodylate buffer with 2.4% sucrose and 8 mM calcium chloride) for 5 minutes and then fixed with 2% osmium tetroxide (OsO₄) in wash buffer for 1 hour at room temperature. Samples were rinsed with DI water to remove unbound osmium, then dehydrated through grade series of ethanol. Subsequently, ethanol was replaced with hexamethyldisilazane (HMDS) and dried at −30° C. The samples were observed with scanning electron microscope (FEI Quanta 600 FEG, FEI, Oregon). For transmission electron microscope (TEM) analysis, samples were fixed with a mixture of 2% paraformaldehyde, 2% glutaraldehyde, 2% OsO₄ in 0.1M sodium phosphate buffer and dehydrated in grade series of ethanol. The samples were then embedded in epoxy resin to cut into 70 nm thickness. The ultrathin sections were observed with a transmission electron microscope (JEOL JEM-1400 Plus, JEOL, Tokyo).

2.6 Cell Viability Assay

Cell viability was measured with live and dead viability/cytotoxicity assay (Thermo Fisher Scientific, Massachusetts). Cell sheet and trypsin treated cell groups were washed twice and incubated with Live/Dead working solution (4 mM ethidium homodimer-1 and 2 mM calcein AM) for 30 minutes at 37° C. in the dark. The samples were washed with and visualized using an AX 10 microscope (Carl Zeiss Microimaging, Gottingen, Germany) and analyzed with Axiovision software (Carl Zeiss Microimaging) (Ex/Em 495/635 ethidium homodimer-1; Ex/Em 495/515 calcein). The number of live and dead cells in single suspension group were counted using image J (National Institutes of Health, Bethesda, Md., USA). The number of dead cells in cell sheet was also counted using image J (National Institutes of Health), whereas live cells in cell sheet was calculated based on the following;

$$Number of live cells in 1 sample = \frac{Area of 1 sample ( {cm}^{2} )}{Total area of cell sheet ( {cm}^{2} )} \times Total cell number$$

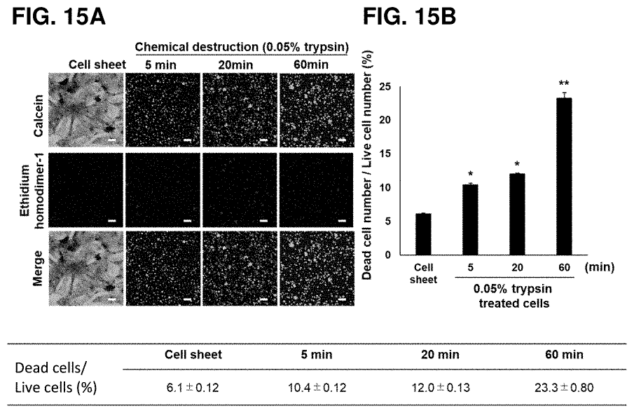

As shown in FIG. 15B, the ratio of dead cells was calculated to compare cell survival rate in each sample.

2.7 Qualitative Analysis of Proteins Related to Cell Functions

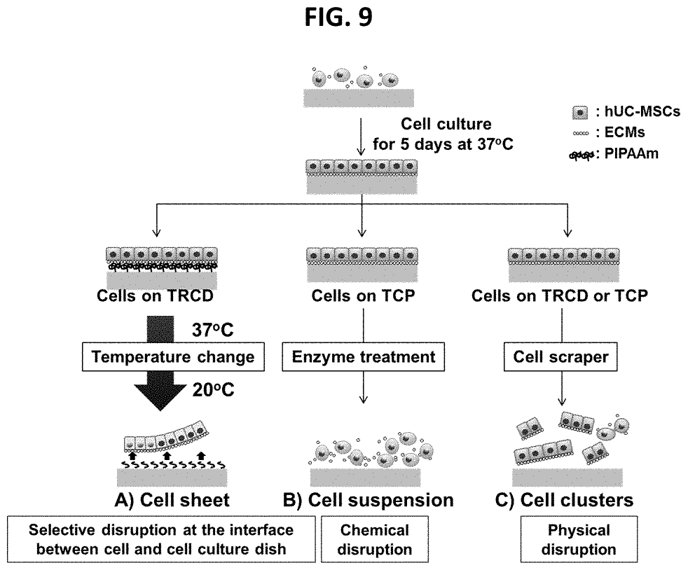

hUC-MSCs (2×10⁵ cells/dish) were cultured for 5 days and harvested by temperature change (cell sheet technology), trypsin treatment (chemical disruption), or cell scraper (physical disruption) (FIG. 9). Cells were lysed with cell lysis buffer (RIPA buffer, proteinase inhibitor and phosphatase inhibitor) (Thermo Fisher Scientific) for 15 minutes at 4° C. to isolate protein extracts. Samples were then sonicated for 9 sec three times. The protein concentration of each sample was determined by Bradford method (Galipeau et al., 2018, Cell Stem Cell 22(6): 824-833). The samples containing same amount of proteins (10 μg) were denatured at 70° C. for 10 minutes and were loaded onto SDS-PAGE gel (3-8% tris-acetate gels or 4-12% tris-glycine gel (Thermo Fisher Scientific)) and transferred electrophoretically to PVDF membranes (LC2002) (Thermo Fisher Scientific). The membranes were treated with blocking solution 5% bovine serum albumin (BSA) for 1 hour at room temperature and incubated with primary antibodies at 4° C. overnight; actin (1:1000 dilution), vinculin (1:10000 dilution), fibronectin (1:2000 dilution), laminin (1:1000 dilution), integrin β-1 (1:2000 dilution), connexin 43 (1:8000 dilution), YAP (1:1000 dilution), phosphor-YAP (Ser127) (1:1000 dilution), FAK (1:1000 dilution), phospho—FAK (Tyr397) (1:1000 dilution), GAPDH (1:5000 dilution). The incubated membranes were treated with appropriate HRP-conjugated secondary antibodies at room temperature for 1 hour. The membrane was visualized by using enhanced chemiluminescence (FluorChem HD2, ProteinSimple, California, USA). The expression levels were normalized to GAPDH.

2.8 Immunocytochemistry Staining of Proteins Related to Cell Functions

Samples were fixed in 4% buffered PFA and then permeabilized with 0.1% triton X-100 (Thermo Fisher Scientific). The samples were blocked with 1% BSA in 10% goat serum for 15 minutes and then incubated in primary antibodies overnight at 4° C.; actin (5 μg/ml), vinculin (1:50 dilution), fibronectin (1:100 dilution), laminin (1:50 dilution), collagen-1 (1:100 dilution), integrin β-1 (1:200 dilution), connexin 43 (1:100 dilution) in the presence of 1% BSA with 10% goat serum. The samples were treated with secondary antibodies for 1 hour. Finally, it was mounted with mounting solution (ProLong Gold Antifade Mountant with DAPI) (Thermo Fisher Scientific) and inspected using IX73 fluorescence microscope (Olympus).

2. 9 Statistical Analysis

All values are expressed as the mean±SEM. Two-way analysis of variance followed by the Tukey's test was used to evaluate differences between more than two groups. Probabilities (p<0.1, 0.05) were considered significant.

Results

Human Umbilical Cord Stem Cell (hUC-MSC) Sheet Preparation

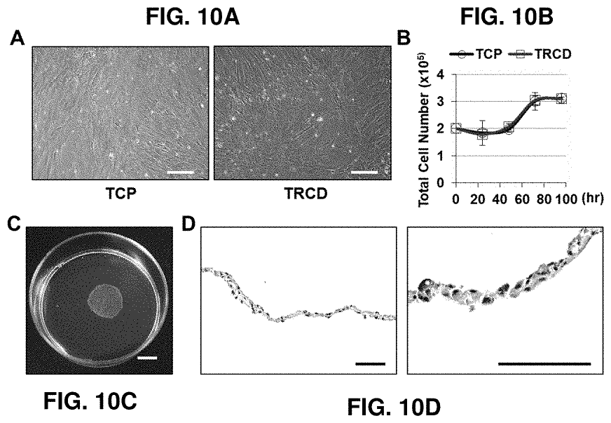

To verify morphologies and growth rates of hUC-MSCs cultured on temperature responsive cell culture dishes (TRCD), hUC-MSCs were seeded at a density of 2×10⁵ cells on conventional tissue culture plates (TCP) or on 35-mm diameter TRCD and were cultured for 5 days. Cells cultured on TRCD have changed its morphology from rounded shape to spindle shape when cells attached to the bottom surface of TRCD. This morphological change was also observed in cells cultured with TCP (FIG. 10A). Additionally, the growth rate of hUC-MSCs cultured on TRCD showed same growth curve with that on TCP (FIG. 10B). This indicates that the cell culture dish surface coated with temperature responsive polymer didn't does not affect cultured cell growth and morphologies of cells. Furthermore, the cells were successfully detached, maintaining a sheet from the TRCD upon temperature decrease from 37° C. to 20° C. (FIG. 10C). The fabricated cell sheets formed comprised a complete, contiguous cell layer, and maintained cell binding proteins similar to native structures (FIG. 10D).

Morphological Observation of hUC-MSC Sheet

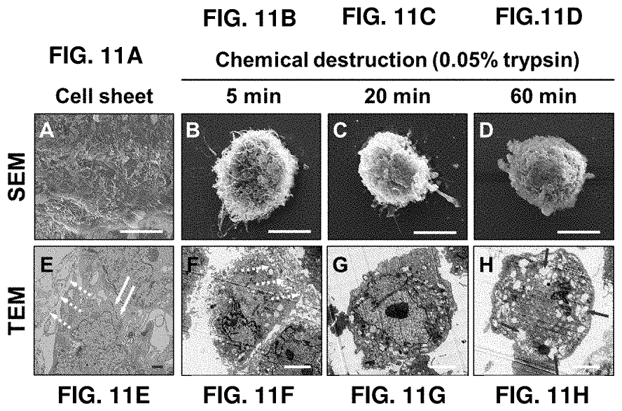

The surface and intercellular structures of hUC-MSC sheets were observed by scanning electron microscopy (SEM) (FIGS. 11A-D) and transmission electron microscopy (TEM) (FIG. 11E-F). In SEM analysis, hUC-MSC sheet s showed connected cell membrane structures on the cell surfaces. It means that the hUC-MSC sheet preserved retention of native structures formed when they are cultured on cell culture dishes, even after cell detachment. Native cellular membrane structure is comprised of cell surface proteins and membrane proteins, which is related to cell adhesion and functions. This finding suggests that hUC-MSC sheets retaining cell surface proteins and membrane proteins and this retention may improve cell adhesion and cell functions (Albuschies et al., 2013, Sci Rep 3: 1658). In contrast, the hUC-MSCs treated with 0.05% trypsin showed dissociated single cell shapes with no connected tissue structures (FIG. 11B-D). In addition, the cell surface in 0.05% trypsin treated groups (5 minutes, 20 minutes, and 60 minutes) lost their microvilli-like structure by trypsin treatment-time dependently (FIG. 11B-D). As a result, hUC-MSC sheets had maintained tissue-like connected structures as well as microvilli-like structures, while proteins on cell surfaces in 0.05% trypsin treated group were cleaved.

In TEM analysis, hUC-MSC sheets maintained ECMs (white dotted line) and cell-cell junctions (white solid arrow), which are related to cell adhesion and cell-cell communication (Gattazzo et al., 2014, Biochim Biophys Acta 1840(8): 2506-19). (FIG. 11E). However, hUC-MSCs treated with 0.05% trypsin for 5 minutes showed cleaved cell-cell junctions and ECMs, compared to the cell sheet group (FIG. 11F). Furthermore, when the hUC-MSCs were treated with 0.05% trypsin for 20 and 60 minutes, hUC-MSCs lost native its filopodia on their cell surface and had unclear nuclear shapes of nucleus (FIGS. 11 G and H). The hUC-MSCs treated with 0.05% trypsin for 60 minutes showed endoplasmic reticulum (dark grey arrows), which is known to associate with cell death (FIG. 11 H). SEM and TEM results indicate that hUC-MSC sheets had maintained cell surface proteins and intercellular proteins such as microvilli-like structures, filopodia, ECM, and cell-cell junctions even after cells were detached from cell culture dish. In contrast, hUC-MSCs treated with 0.05% trypsin groups showed cleaved microvilli, ECM, and cell-cell junctions and damaged the nucleus. The findings suggest that trypsin treatment (chemical disruption) causes damage in cell and tissue structures (i.e. junction proteins, ECMs, nucleus, and endoplasmic reticulum).

hUC-MSC Maintains Actin Filament Proteins Relating with Cell Dynamics

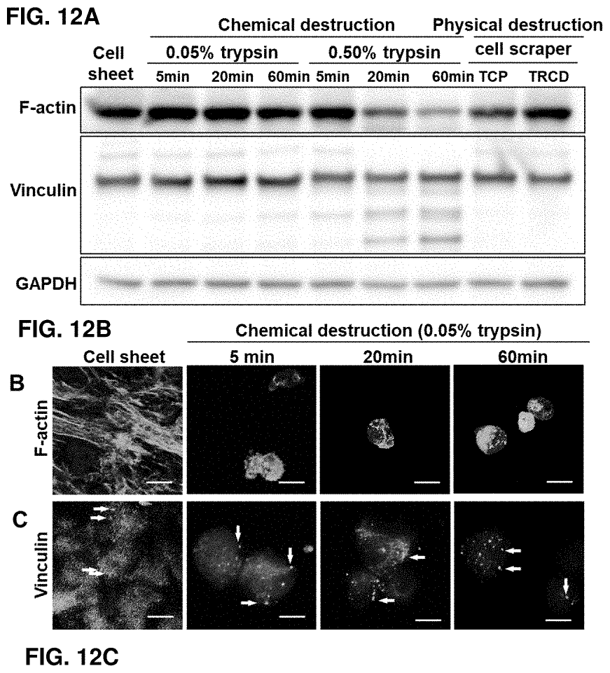

Glyceraldehyde 3-phosphate dehydrogenase (GAPDH) protein expression was detected as a loading control to normalize protein amounts for western blotting assay. GAPDH protein expression level was similar in all groups. Cells treated with 0.50% trypsin for 20 and 60 minutes expressed lower actin than that in cell sheet, 0.05% trypsin, and cell scraper groups (FIG. 12A). This indicates 0.50% trypsin treatment disrupts actin in cytoplasm. To observe cytoskeleton structure, hUC-MSCs were stained with actin. When cells are attached to culture dish, actin forms stress fiber structure which plays an important role in cell survival (Bachir et al., 2017, Cold Spring Harb Perspect Biol 9(7)). Cell sheet groups showed exhibited actin stress fiber structures of actin even after the cell sheet was detached from cell culture dish. In contrast, 0.05% trypsin-treated groups for 5, 20 and 60 minutes showed actin positive area, however stress fiber structures were not observed (FIG. 12B). The amount of F-actin protein was similar in cell sheet and 0.05% trypsin treated groups. However, only cell sheet groups maintained actin stress fiber structures characteristic of cell actin.

Vinculin is a membrane cytoskeletal protein that forms focal adhesion by linking integrin family and actin, associated with cell movement (Peng, 2011, Int Rev Cell Mol Biol 287: 191-231). Vinculin expression was observed in both cell sheet and 0.05% trypsin treated groups when stained with immunohistochemistry (FIG. 12C). Multiple lower molecular weight bands in western blot analysis of vinculin expression were observed in the chemical disruption group (FIG. 12A). This indicates that vinculin proteins were cleaved in the chemical disruption group. The cells treated with trypsin (chemical disruption) revealed delocalized actin fiber structures, reduced actin protein, and cleaved vinculin protein, suggesting chemical disruption method cleaved proteins related to cell shape and cell dynamics. This cleavage was increased when trypsin concentration was increased.

hUC-MSC Sheet Maintains Extracellular Proteins Related with Cell Adhesion

Fibronectin and laminin are important proteins in cell- and tissue-adhesion. Cell sheet, 0.05% or 0.50% trypsin treatment for 5 minutes, and cell scraper groups in western blot assay expressed fibronectin. However, 0.05% and 0.50% trypsin treatment for 20 minutes, and 60 minutes groups had no detectable expression of fibronectin. Laminin expression was observed in cell sheets, 0.05% trypsin treatment, 0.50% trypsin treatment for 5 minutes, and cell scraper groups. However, 0.50% trypsin treatment groups for 20 minutes and 60 minutes showed no detectable laminin expression.

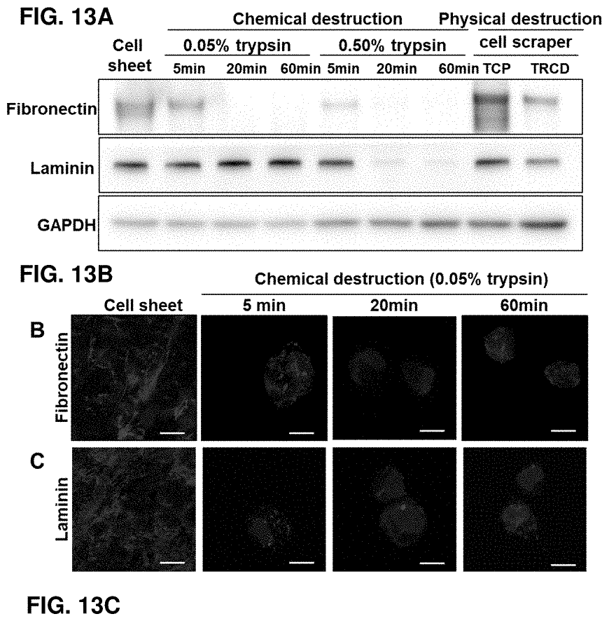

Cells were stained using fibronectin and laminin antibodies to observe the ECM protein structures of ECM proteins (FIG. 13B). Higher expression of fibronectin was observed in the cell sheet group, compared to cells treated with 0.05% trypsin. Cell sheet groups showed higher expression of fibronectin and laminin over all cells in the cell sheet, similar to native tissue structures (fibrous structure of ECM). These results suggest that the cell sheet group was able to detach cells without disruption of ECM. In contrast, ECM proteins were cleaved with trypsin treatment (chemical disruption) after cell detachment from cell culture dishes.

hUC-MSC Sheet Maintains Cell Junction Proteins Associated with Cell Communication

Integrin β-1 is a major protein of the integrin family of transmembrane membrane proteins that forms cell-ECM junction. It is known that integrin links to the cell's actin cytoskeleton through adapter proteins (e.g. vinculin, talin) and is involved in cell phenotypic preservation, survival, cell adhesion and tissue repair (Moreno-Layseca, 2014, Matrix Biol 34: 144-53. Cell sheet, 0.05% trypsin treatment for 5 minutes and cell scraper groups showed similar Integrin β-1 expression. Integrin β-1 was cleaved gradually as with trypsin concentration and treatment time. Connexin 43 is a transmembrane protein that in gap junctions that facilitates cell-cell communication. Connexin 43 plays essential role in maintaining homeostasis and function of cells and tissues by exchange of biological information (Ribeiro-Rodrigues, 2017, J Cell Sci 130(21): 3619-3630). Connexin 43 was expressed in the cell sheet, 0.05% trypsin treated (5, 20, 60 minutes) and 0.5% trypsin treated (5 minutes) group. However, 0.50% trypsin treatment for 20 and 60 minutes had no expression of Connexin 43. This suggests that Connexin 43 protein was cleaved by 0.50% trypsin when treated for 20 and 60 minutes.

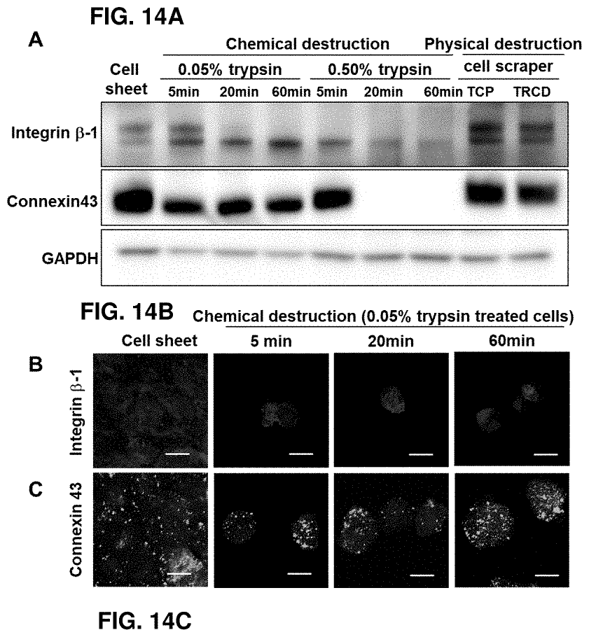

Structural observation of cell junction proteins was performed with Integrin β-1 and Connexin 43 protein was observed by immunostaining. Cell sheet group showed positive expression of Integrin β-1 all uniformly over the cell sheet, whereas Integrin β-1 was expressed slightly on the cell surface in 0.05% and 0.50% trypsin treated groups (FIG. 14B). Connexin 43 expression was observed in all groups (FIG. 14C). Especially, cell sheet groups revealed Connexin 43 expression in all over the visualized the area. This demonstrates that cell sheets have connected tissue cell-cell structures and maintains cell-junction proteins. In contrast, trypsin treatment (chemical disruption) cleaved these important junction proteins.

Chemical Disruption Method Induces Cell Death

Cells were stained using calcein and ethidium homodimer-1 immediately after cell detachment by trypsin treatment (chemical disruption) or temperature changes (cell sheet technology). Green color shows live cells and red color shows dead cells in FIG. 15. For results, dead-cell to live-cell ratios in 0.05% trypsin treatment for 5 and 20 minutes groups were similar. Additionally, dead-to-live cell ratios for cells in 0.05% trypsin treatment for 60 minutes groups significantly increased, compared to cells treated with 0.05% trypsin for 5 and 20 minutes (FIG. 15B). This result suggests that cell death was induced by trypsin treatment (chemical disruption).

Apoptotic Cell Death is Activated by Chemical Disruption

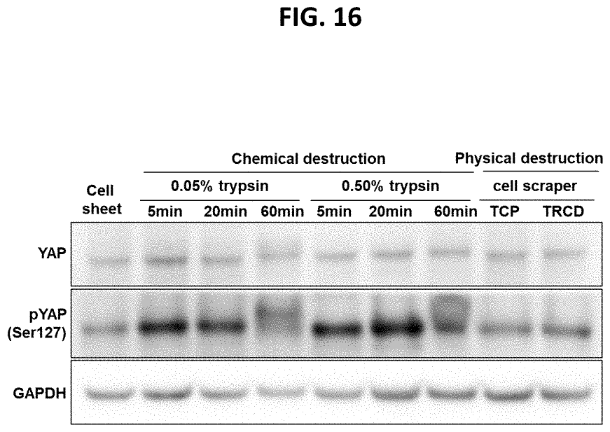

Mechanosensor controls for cellular homeostasis convert extracellular physical stimuli to intracellular chemical stimuli (Humphrey, 2014, Nat Rev Mol Cell Biol 15(12): 802-12). Yes-associated protein (YAP) is a major mechanosensor protein localized at cell nuclei to regulate cell survival and proliferation (Jaalouk, 2009, Nat Rev Mol Cell Biol 10(1): 63-73). YAP is inhibited via phosphorylation of Ser127 (phosphor-YAP, pYAP), which results in cytoplasmic retention and induction of cell apoptosis. When cells lose cell-ECM junctions, apoptotic cell death, namely anoikis, is induced subsequently to phosphorylation of YAP (Halder et al. 2012, Nat Rev Mol Cell Biol 13(9): 591-600). YAP and phospho-YAP (pYAP) expression of cell sheets, 0.05% and 0.50% trypsin treatments for 5, 20 and 60 minutes and in the cell scraper group were determined with western blotting (FIG. 16). All groups showed similar YAP protein expression, whereas expression of pYAP was increased in 0.05% and 0.50% trypsin treated cells compared to cell sheet and cell scraper group (FIG. 16A). This demonstrates that trypsin treatment (chemical disruption) inhibited YAP activity and induced phosphorylation of YAP. In addition, induction of pYAP is known to shift cell responses towards apoptosis.

Discussion

Chemical disruption methods are commonly used to harvest cells from cell culture dishes through disruption of extracellular (Huang et al., 2010, J Biomed Sci 17: 36) and intercellular (Besingi, 2015, Nat Protoc 10(12): 2074-80) proteins associated with the cytoskeleton, cell junctions, cell metabolism, and cell growth. Hence, cells harvested by chemical disruption methods (e.g., trypsin-treated cells) had insufficient ECM which are necessary to adhere to target tissues, and insufficient cell junctions to maintain their normal cellular functions through graft-host communication (FIGS. 13 and 14). On the other hand, hUC-MSC sheets harvested by cell sheet technology using TRCD maintained tissue-like structures including smooth surfaces of connected cells, microvilli, ECM, and cell-cell junctions (FIGS. 10, 13, and 14).

TEM results showed that extracellular protein cleavage was observed in cells treated with 0.05% trypsin for 5 minutes in the chemical disruption group. Cytoplasm cleavage was observed in after 20 minutes of 0.05% trypsin treated cells; and cell nucleus has degraded at 60 minutes of 0.05% trypsin treatment. In addition, endoplasmic reticulum changes related to cell death were observed at 60 minutes of 0.05% trypsin treatment (FIG. 11). Integrin is a key protein involved in the interaction between cell membranes and ECMs, also linking ECMs to cytoskeleton (actin) to form focal adhesions (Kim et al., 2011, J Endocrinol 209(2): 139-51). Harvest by chemical disruption induced cleavage of integrin β-1 as well as cytoskeleton (F-actin), focal adhesion protein (vinculin), ECMs (fibronectin and laminin) (FIGS. 12, 13, and 14). On the other hand, hUC-MSC sheet maintained all integrin β-1, cyto skeleton, focal adhesion protein (vinculin) and ECMs (fibronectin and laminin) even after detachment from cell culture dishes (FIGS. 12, 13, and 14). These findings suggested that chemical disruption methods (e.g., proteolytic trypsin treatment) may cause a harsh environment for compromising cell survival due to proteolytic cleavage of membrane receptors and disruption of cell-ECM junctions by enzyme.

Yes-associated protein (YAP) has an important role in regulating cell adhesion, proliferation and survival. It is known that apoptotic cell death is induced through inhibition of YAP and subsequent pYAP induction. Similarly, breakdown of cell-ECM junction induces apoptotic cell death through inhibition of YAP (Codelia, 2012, Cell 150(4): 669-70). When cells were treated with trypsin (chemical disruption), integrin β-1 was cleaved (FIG. 14) and the cleavage of integrin β-1 inactivated YAP and induced pYAP (FIG. 16). Eventually, apoptotic cell death occurred in the chemical disruption group (FIGS. 11, 15, and 16). In contrast, the hUC-MSC sheets maintained integrin β-1 and reduced expression of pYAP (FIGS. 14 and 16) which then showed significantly higher cell survival rates (FIGS. 15 and 16). It is reported that pYAP can be induced by not only integrin β-1 cleavage but also inhibition of F-actin polymerization. The hUC-MSC sheet showed cytoskeleton fiber structures of F-actin indicating active actin polymerization even after cell detachment from cell culture dishes (FIG. 12). This suggests that hUC-MSC sheets retains integrin β-1 (cell-ECM junction) and fiber structures of F-actin fibers, allowing cell sheets to maintain higher cell survival rates compared to trypsin treatment (conventional chemical disruption cell harvesting method). These findings suggest that cell-ECM junction and actin fiber maintenance structures are important factors for maintaining cell survival. High cell survival rates are difficult using chemical disruption methods.

This study demonstrates that ECM, cell-cell junction and cell-ECM junction proteins are important in retaining higher cell survival rates. In conventional stem cell therapies that use chemical disruption harvesting methods, it is not possible to avoid low engraftment rate efficiencies and low cell survival rates, since chemical disruption (e.g. trypsin treatment) cleaves cell-cell junctions, cell-ECM junctions and cell adhesion proteins. Cell sheet technology enables cells to be harvested in a viable sheet form without any structural disruption. Furthermore, cell sheet technology maintains important structures for cells (ECMs, cell-ECM junctions, cell-cell junctions, cytoskeleton and mechanosensors) enhancing cell survival rates, engraftment efficiencies and maintaining various critical cellular functions. As a result, cell survival rates in hUC-MSC sheets are significantly higher than for cells harvested with chemical disruption methods.

Conclusion

We demonstrate that retaining tissue-like structure such as ECMs cell-cell junction and cell-ECM junctions are associated with enhanced cell survival rates for of transplanted cells. Cell sheet technology allows harvest of cells in sheet form without using any damaging proteolytic enzymes (chemical disruption). Harvested hUC-MSC sheets that retain tissue-like cell structures, ECMs, cell-cell junctions and cell-ECM junctions had higher cell survival rates, compared to conventional chemical disruption methods (trypsin treatment). This technology will provide not only a higher therapeutic effect of stem cell therapy, but also new concepts for improving cell functions in regenerative medicine research since cell sheets mimic native tissue-like structures.

### Example 3. Gene Expression in Human Umbilical Cord Mesenchymal Stem Cell (hUC-MSC) Sheets

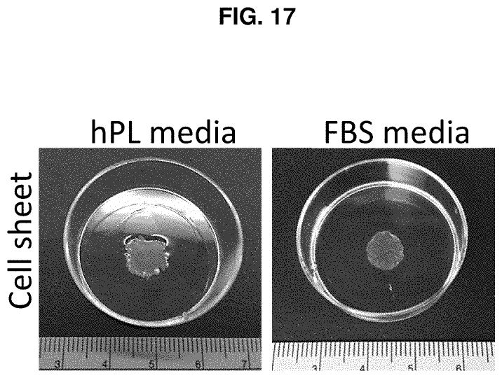

Cell sheets were prepared from hUC-MSCs by the methods described in Example 1 above, except that the cell culture medium contained either 20% hPL or 20% FBS. The hUC-MSC sheets are shown in FIG. 17. Single cell suspension cultures of hUC-MSCs were prepared by culturing hUC-MSCs on cell culture dishes and treating the cells with trypsin (TryLE, Gibco) when they were confluent. The trypsinized single cell suspensions of hUC-MSCs were analyzed by flow cytometry.

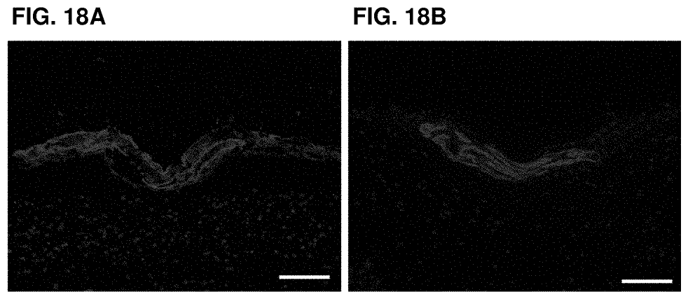

hUC-MSC sheets were cultured in medium containing 20% hPL and implanted within the subcutaneous tissue of immuno-deficient mice as described in Example 1 above, and the hUC-MSC sheets were harvested from the subcutaneous tissue sites for histological observation at 1 day and 10 days after implantation. After harvest, the samples were stained with human growth factor (HGF) antibody for detection of HGF expression, and cell nuclei were stained with DAPI. As shown in FIG. 18, the hUC-MSC sheets expressed HGF 1 day after implantation, and still maintained significant HGF expression 10 days after implantation. These results suggest that hUC-MSC sheets maintain continuous HGF expression for at least 10 days after implantation into the tissue of a host organism.

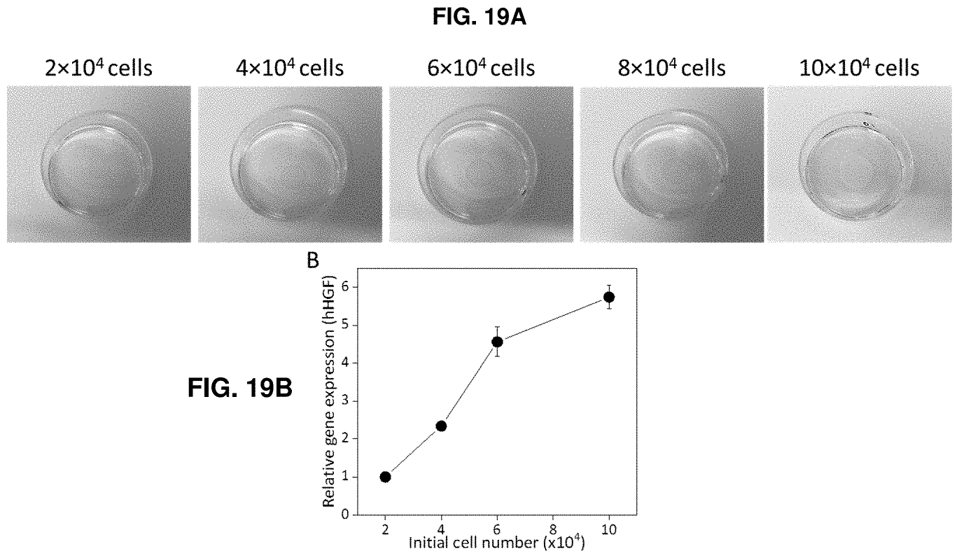

The effect of initial cell density on HGF expression in hUC-MSC sheets was also determined. Cell sheets were prepared from hUC-MSCs in TRCD with an initial cell density of 2×10⁴, 4×10⁴, 6×10⁴, 8×10⁴ or 10×10⁴ cells/cm² in cell culture medium containing 20% FBS. As shown in FIG. 19, increasing the initial cell density increased HGF expression in a cell density-dependent manner. For example, the cell sheets produced with 1×10⁵ cells/cm² had higher HGF gene expression, compared to the cell sheets produced with 2×10⁴, 4×10⁴, 6×10⁴, 8×10⁴ or 1×10⁵ cells/cm². These results suggest that HGF expression levels in the hUC-MSC sheet can be optimized by controlling the initial cell density in the cell culture support (e.g. the TRCD).

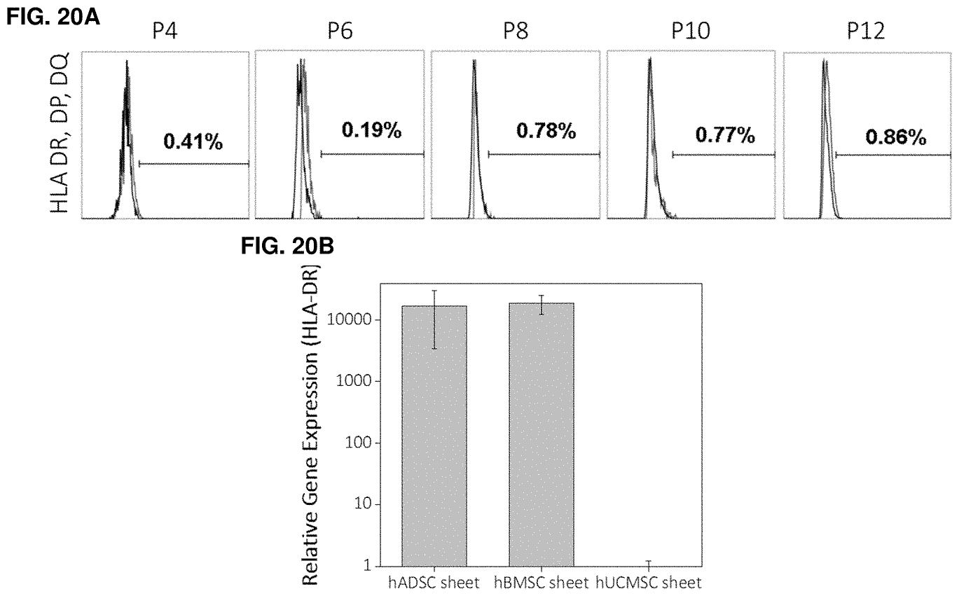

HLA DR, DP, DQ expression was determined in hUC-MSCs in suspension cultures from passage 4 to 12, and in cell sheets prepared from human adipose-derived mesenchymal stem cells (hADSC), human bone marrow-derived mesenchymal stem cells (hBMSC), or hUC-MSCs. Cells were grown in culture media containing 20% hPL. HLA expression was determined as described above in Example 1. As shown in FIG. 20A, hUC-MSCs maintained low HLA DR, DP, DQ cell surface expression from passage 4 to 12 in cell suspension cultures. As shown in FIG. 20B, HLA-DR gene expression was not detectable in hUC-MSC sheets, while cell sheets prepared from hADSC or hBMSC exhibited relatively high levels of HLA-DR gene expression. Low HLA expression is desirable for reducing host immune response to huC-MSC cell sheets transplanted to a host organ. Accordingly, these results suggest that hUC-MSC sheets are less likely to induce an immune response in a host organism after transplantation relative to cell sheets produced from hADSCs or hBMSCs.

### Example 4. Transplantation of a Human Umbilical Cord Mesenchymal Stem Cell (hUC-MSC) Sheet Prevents Uterine Scar Development in a Nude Rat Model

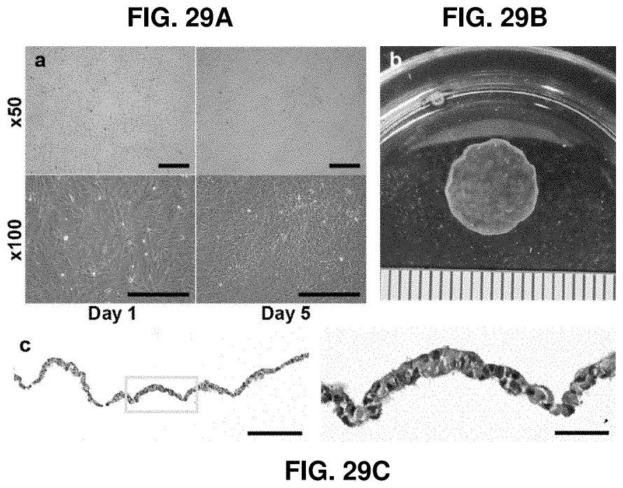

Human umbilical cord mesenchymal stem cells (hUC-MSCs) were used for making cell sheets as described in Example 1 above using an initial cell density of 3.0×10⁵ in a thermo-responsive cell culture dish (TRCD) (UpCell®, Tokyo, Japan). Images of the cell sheets are provided in FIG. 29A-29C. Commercial human umbilical cord mesenchymal stem cells used in prior human clinical trials were cultured on commercial temperature-responsive cell culture dishes and allowed to proliferate to confluence on day 5 (FIG. 29A). Cell sheets were naturally harvested at reduced culture temperature (20° C.) without destructive proteolytic enzymes over 30 minutes (FIG. 29B). HE staining evidenced a contiguous cell sheet with regular, tight cell-cell connections and single and bi-layered cuboidal cells comprising the cell sheet (FIG. 29C). All seeded temperature-responsive cell culture dishes resulted in cell sheets, confirming the feasibility of the method used. The resulting human umbilical cord mesenchymal stem cell sheets could then be transferred to rat uterine transplantation sites using a thin plastic spatula (FIG. 29B).

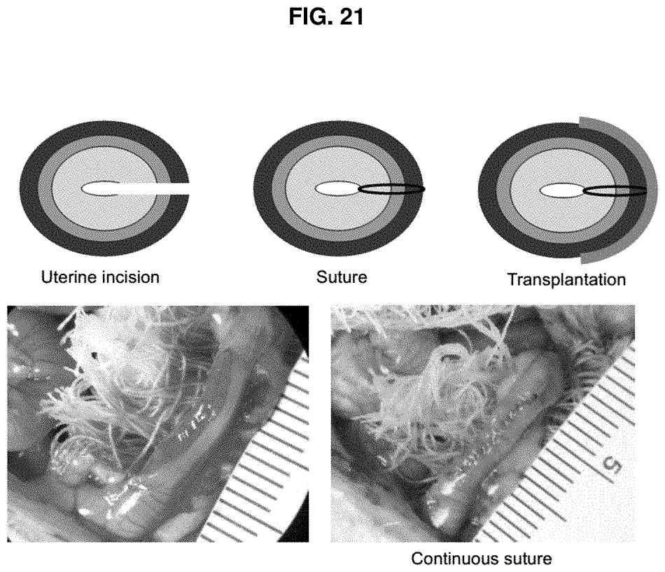

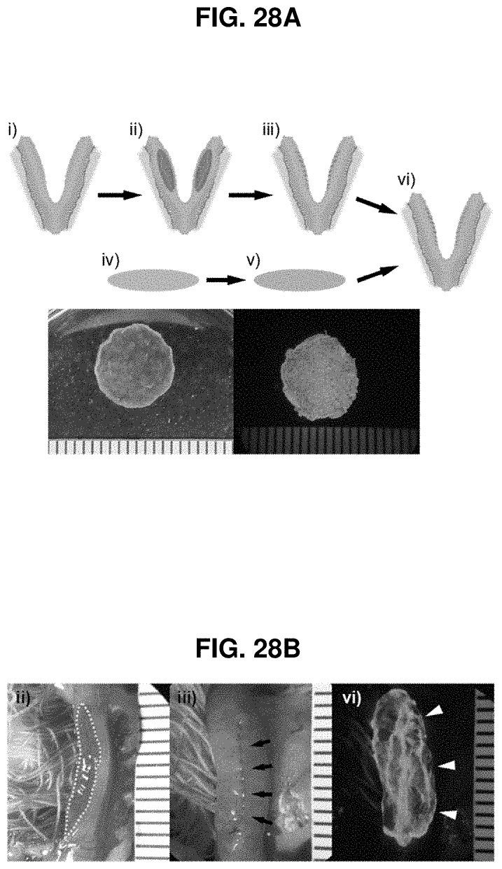

After harvesting, the cell sheet was labeled with a fluorescent marker, carboxyfluorescein succinimidyl ester (CFSE), to allow the cell sheet to be identified after transplantation. Uterine incision was performed to induce scar formation using non-pregnant female nude rats (FIG. 21). A schematic and photographs of the cell transplantation process are provided in FIG. 28A-28B.

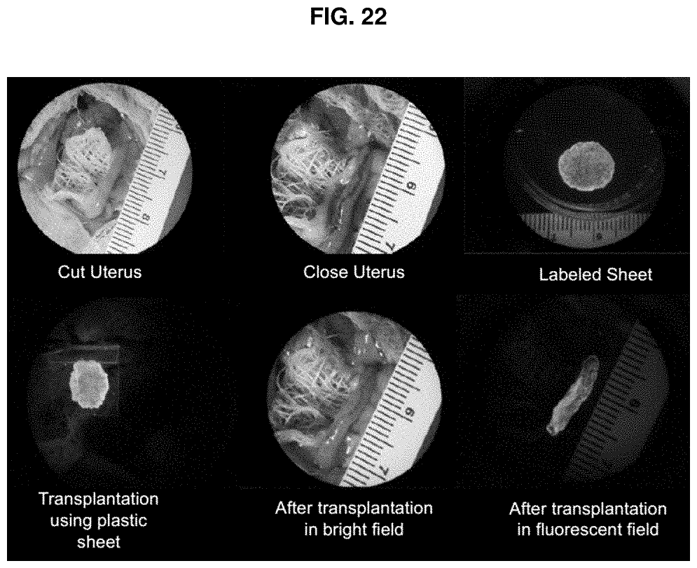

Longitudinal incisions were made on each horn of the didelphic rat uterus. After suture repair of both uterine incisions, a hUC-MSC sheet was transplanted to the surface of the hysterotomy repair of one horn of the uterus, while the contralateral horn of the uterus served as a control. In the transplantation group, a hUC-MSC sheet approximately 1 cm² in surface area was transplanted to the uterus, and the position of the transplanted cell sheet was confirmed by fluorescent microscopy as a green colored area (FIG. 22). For transplantation, the cell sheet was floated in medium in the cell culture dish and then transferred to a square shaped plastic sheet approximately 1 cm across. The cell sheet was transported to the uterus on the plastic sheet. The cell sheet was transferred to the uterine wound by sliding it down from the plastic sheet with forceps (FIG. 22). The shape and adhesive extracellular matrix of the cell sheet allowed it to be fixed directly to the uterine wound without any scaffolds or sutures.

Proper positioning and location of the cell sheet was confirmed by fluorescent microscopy and evaluated on days one, three, and seven after surgery. At 1, 3, 7 and 14 days post-surgery, uteri were harvested for macroscopic analysis and histological evaluation by hematoxylin and eosin (HE) staining and Masson-Trichrome staining. The fibrotic (blue) and normal (purple/red) myometrium surface was analyzed using AmScope® software, and the ratio of fibrotic-to-normal myometrial surface area was calculated for each specimen.

Expression of human hepatocyte growth factor (HGF) and vascular endothelial growth factor A (VEGFA) was measured by quantitative real-time PCR. Scars in both control and cell sheet-transplanted horns were harvested and frozen in liquid nitrogen on day 1, 3, 7 and 14 (n=3 horns per group). RNA was then extracted from each sample using Rneasy® Fibrous Tissue Mini Kit (74704, Qiagen, Germany) after mincing each horn with a mechanical homogenizer. Using a High Capacity cDNA Reverse Transcription Kit (4368814, Thermo Fisher Scientific), cDNA was synthesized and subjected to PCR analysis (StepOnePlus™ Real-Time PCR System, 4376600, Thermo Fisher Scientific)) using TaqMan® Gene Expression Assays (4331182, Thermo Fisher Scientific) and PCR probes specific to each target gene.

Results

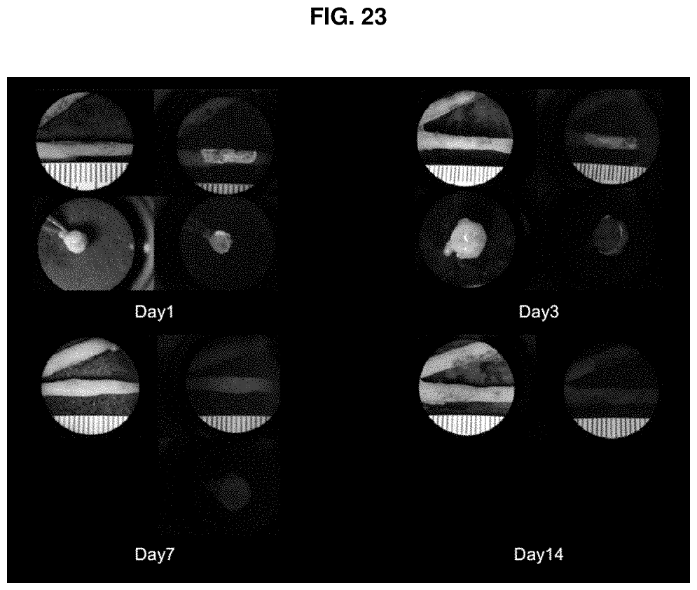

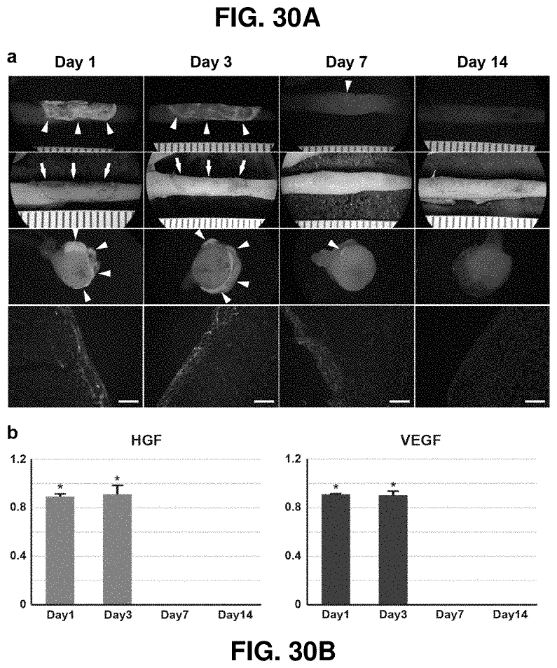

Eighteen uterine specimens from six rats were included in this analysis, 2 or 4 from each horn of the uterus. At 1, 3, and 7 days after transplantation, the presence of the hUC-MSC sheet transplanted to the uterine wound was confirmed by fluorescent microscopy (FIGS. 23 and 28A). Cell sheets stably adhered to the horn wound site surface spontaneously after 30 minutes without suturing. A group of uteruses were harvested on days 1, 3, 7, and 14 post-surgery. Transplanted cell sheets were readily detected in situ on days 1 and 3 by direct fluorescent microscopy observation. However, fewer fluorescent cell sheet fragments were noted on day 7. By day 14, the cell was not observable (FIG. 30A, top row). The borders between the transplanted cell sheet and the uterine surface were well-demarcated at day 1 and 3, but became less clear on day 7 and 14 (FIG. 30A, second and third rows). Green stained cells were present in the histological samples on days 1, 3 and 7, but not 14. Stained cell presence continuously decreased, and on day 14, was no longer evident (FIG. 23 and FIG. 30A, bottom row).

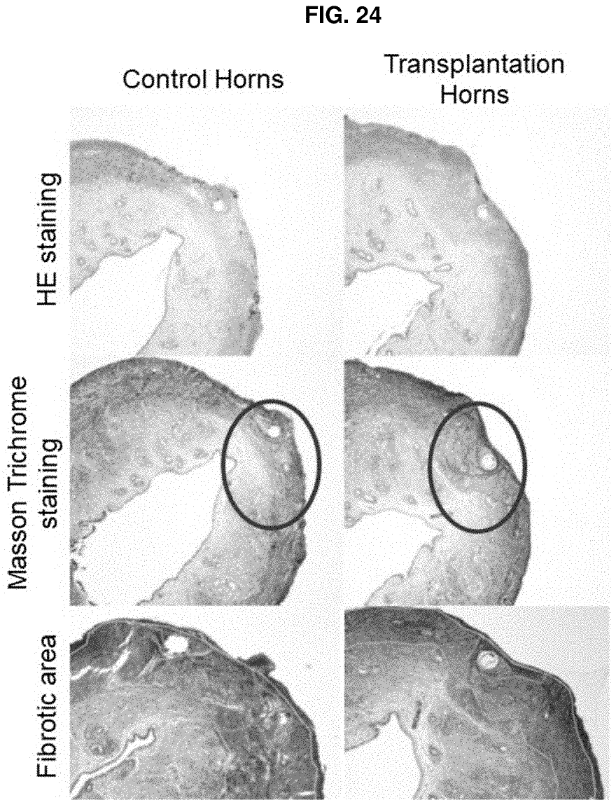

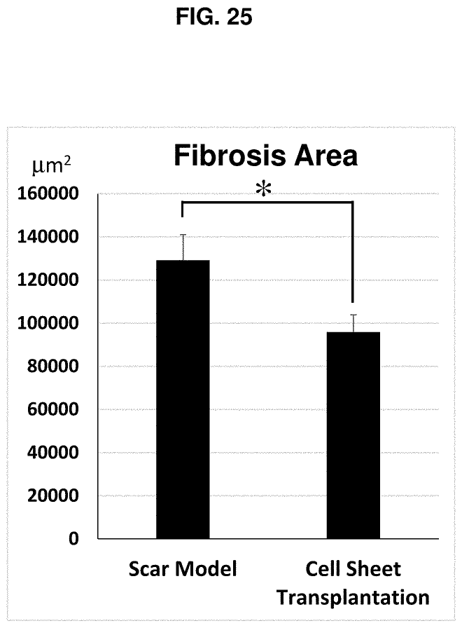

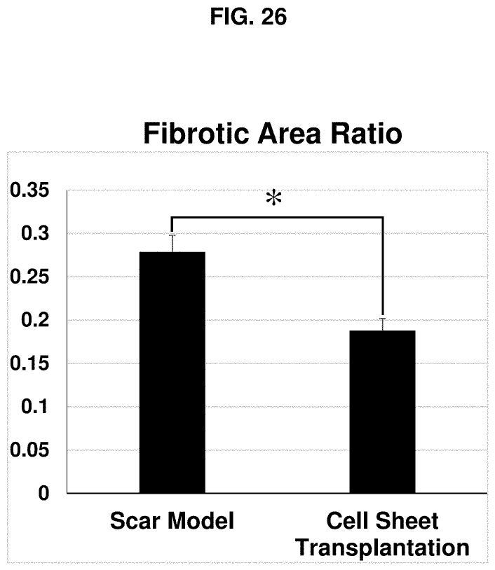

In the control group, a large fibrotic area was present between the host myometrium areas as a result of wound healing. In contrast, the fibrotic area in the hUC-MSC sheet transplantation group was significantly smaller than in the control group (FIG. 24). Specifically, the mean fibrotic surface area in the control group (n=18) was 129185.7 μm² (range 60838.1-245836.1 μm²), and in the hUC-MSC sheet transplant group (n=18) was 95861.6 μm² (range 47090.5-154446.7 μm²) (FIG. 25). Accordingly, application of the hUC-MSC sheet to the uterus reduced the fibrotic surface area of the uterus by approximately 27% relative to the control group in which the hUC-MSC sheet was not applied. The calculated mean ratios of fibrotic-to-normal myometrial surface in controls and the hUC-MSC sheet transplant group were 0.28 and 0.19 respectively (p<0.01) (FIG. 26). Accordingly, application of the hUC-MSC sheet to the uterus reduced the ratio of fibrotic-to-normal myometrial surface by greater than 33% relative to the control group in which the hUC-MSC sheet was not applied. Thus, transplantation of the hUC-MSC sheet significantly reduced fibrosis on the myometrial surface.

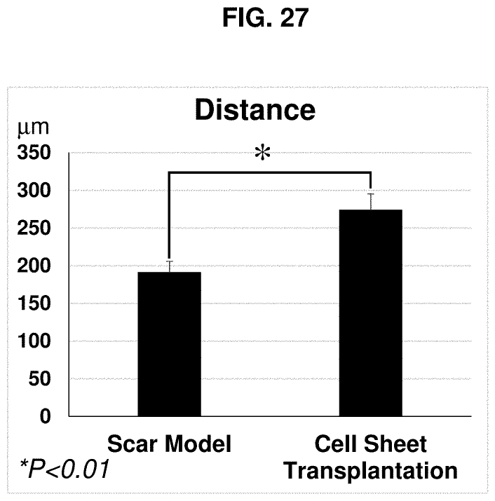

The thickness in incision areas in both control (n=18) and the hUC-MSC sheet transplant group (n=18) were 191.5 μm (range 90.0-296.4 μm) and 274.3 μm (range 143.7-448.4 μm) respectively (p<0.01) (FIG. 27).

Gene expression of human hepatocyte growth factor (HGF) and human vascular endothelial growth factor (VEGF) from cell sheet-transplanted uteruses was measured at 1, 3, 7 and 14 days after transplantation. The gene expression of human HGF message and human VEGF message from scarred control uterus were not detected on all days. Human gene expression differences between the cell sheet transplantation and control groups are significant at days 1 and 3 (*p<0.01). Scarred control uterine gene expression was not detectable, and not shown.

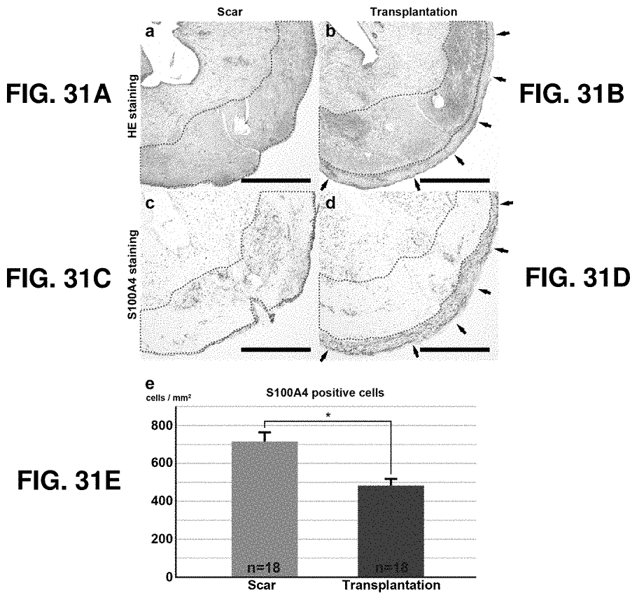

Numbers of fibroblast cells in myometrial tissues were detected using immunostaining for S100A4 protein, specific for fibroblasts (Kong, et al., 2013, Am J Physiol Heart Circ Physiol. 305(9): p. H1363-72). The border of the myometrial (dotted line), endometrial lining, and transplanted cell sheet areas (arrows) were identified with HE-stained specimens. S100A4-positive cells were counted in myometrial areas using fibroblast specific protein-specific immunostaining. The mean number of fibroblasts in cell sheet transplanted horns was 483/mm² (SD: 137/mm²) compared to 716/mm² (SD: 194/mm²) in control horns (FIG. 31B). The number of S100A4-positive cells in the control horns is significantly higher than that in the cell sheet transplantation group (p=0.0002).

Gene expression of human hepatocyte growth factor (HGF) and human vascular endothelial growth factor A (VEGFA) were detected on days 1 and 3 and were higher in cell sheet-transplanted horns compared to control horns. However, these genes were not expressed on days 7 and day 14 (FIG. 30B).

Discussion In an immune-deficient rat hysterotomy model, we demonstrated that the transplantation of human umbilical cord mesenchymal stem cell sheets onto the hysterotomy at the time of surgical repair is feasible, as measured by the development of appropriately sized sheets with natural adhesion and by retention of the stem cells in the target area by immunohistochemistry after several days. We further demonstrated that stem cell sheet transplantation stimulates improved wound healing, as demonstrated by reduced fibroblast recruitment, decreased fibrosis formation, increased postoperative myometrial thickness, and improved ratio of normal myometrium to fibrotic tissue in the area of the hysterotomy.

This study used human umbilical cord-derived stem cells that have been previously used for treating human heart failure as clinically injected cell suspensions. Previous work has shown that these human umbilical cord mesenchymal stem cell sheets cultured in fetal bovine serum (FBS) express low major histocompatibility complex (MHC) class II antigens and maintain low MHC class II antigen expression during cell sheet preparation. These human umbilical cord mesenchymal stem cells express not only normal MHC class I genes, but also non-canonical class I MHC genes (Human Leukocyte Antigen [HLA]-E, HLA-F, and HLA-G) (La Rocca, et al., 2009, Histochem Cell Biol, 131(2): p. 267-82). MHC class I antigens can serve to protect cells from some natural killer cell-induced killing processes. All three non-canonical class I MHC proteins have been reported to be expressed by extravillous trophoblasts, and are associated with maternal tolerance to the semi-allogeneic embryo.

Clinical stem cell preparations are most often administered as injected suspensions. Injected stem cell suspensions exhibit weak, transient short-term cytokine secretion (i.e., less than 3 days) (Elman, et al., 2014, PLoS One, 9(2): p. e89882), and very low tissue engraftment efficiency, decreasing their local effects. However, the cell sheet transplants described herein engrafted locally, spontaneously, rapidly, and efficiently, promoting maximal therapeutic effects at the transplant site without migration. Hence, these human umbilical cord mesenchymal stem cell sheets exhibit reliable phenotypic traits, tissue engagement, and structural features deemed important for human umbilical cord mesenchymal stem cell for host immune histocompatibility, optimal therapeutic and reliable engraftment processes.

This study expands the use of human umbilical cord mesenchymal stem cell sheets to uterine surgical applications, demonstrating feasibility. Cell sheets constructed from these stem cells, in contrast to infused or injected stem cells, retain critical cell adhesive proteins related to therapeutic and engraftment efficacy, including cell-cell junction proteins associated with cell-cell communication and extracellular matrix proteins related to cell adhesion, and cytokine hepatocyte growth factor (HGF) (Kim, et al., 2019, Sci Rep, 2019. 9(1): p. 14415). These cell sheets could be optically tracked for up to 7 days post-transplant (c.f., FIG. 30). Human umbilical cord mesenchymal stem cell sheet transplantation was associated with increased uterine wall thickness, decreased wound site fibrosis, and reduced wound fibroblast cell presence. Human hepatocyte growth factor (HGF) and human vascular endothelial growth factor (VEGF), both important for wound healing and tissue regeneration, were detected in human umbilical cord mesenchymal stem cell sheet transplant horns in early healing phases compared to control horns (FIG. 30). These findings suggest that human umbilical cord mesenchymal stem cell sheet transplantation improves wound healing in nude rat hysterotomy. Our data suggest that human umbilical cord mesenchymal stem cells applied as durable living sheets are promising for hysterotomy repair.

The precise mechanisms for how transplanted stem cell sheets promote myometrial regeneration are uncertain. However, an essential stem cell function is their paracrine effects, through secretion of cytokines and chemokines, including plasminogen activator inhibitor-1 (PAI-1), macrophage migration inhibitory factor (MIF), and interleukin-6 (IL-6), which decrease inflammation, HGF and others to promote regeneration and proliferation of host tissue, and with VEGF, promote angiogenesis. Previous studies have shown that transformation of transplanted stem cells into myometrial tissue is unlikely to be a significant contributor to repair, at least not in the acute phase (Ho, C. H., et al., 2018, J Chin Med Assoc, 81(3): p. 268-276). Further, transplantation of allogenic myometrial cells did not improve hysterotomy wound healing in an animal uterine repair model (Ho et al., 2018, ibid).

Noted reductions in both wound site fibrotic surface area as well fibroblast abundance in this model can be attributed to intrinsic human umbilical cord mesenchymal stem cell sheet anti-inflammatory effects Inflammatory cell mediators (monocytes and macrophages) are attenuated with human umbilical cord mesenchymal stem cell sheet transplantation (La Rocca, et al., 2009, Histochem Cell Biol, 131(2): p. 267-82).

Conclusion

These results demonstrate decreased formation of fibrotic tissue and increased myometrial regeneration following hUC-MSC sheet transplantation onto repaired hysterotomies of rat uteri relative to a control without hUC-MSC sheet transplantation. Accordingly, the hUC-MSC sheets described herein improve healing of the uterine scar and have the potential to decrease morbidities related to abnormal uterine scar formation.

We claim:

1. A method of reducing formation of fibrotic tissue in a uterus of a subject in need thereof, comprising applying a mesenchymal stem cell (MSC) sheet to the uterus of the subject, wherein the MSC sheet comprises one or more layers of aggregated confluent mesenchymal stem cells (MSCs), and wherein applying the MSC sheet to the uterus reduces the formation of fibrotic tissue in the uterus relative to a uterus in which the MSC sheet is not applied.

2. A method of increasing myometrial regeneration in a uterus of a subject in need thereof, comprising applying a mesenchymal stem cell (MSC) sheet to the uterus of the subject, wherein the MSC sheet comprises one or more layers of aggregated confluent mesenchymal stem cells (MSCs), and wherein applying the MSC sheet to the uterus increases myometrial regeneration relative to a uterus in which the MSC sheet is not applied.

3. The method of claim 1, wherein the MSC sheet is applied to an incision site in the uterus.

4. The method of claim 1, wherein applying the MSC sheet to the uterus reduces fibrotic surface area of the uterus by at least 20% relative to a uterus in which the MSC sheet is not applied.

5. The method of claim 1, wherein the MSC sheet consists essentially of MSCs.

6-7. (canceled)

8. The method of claim 1, wherein the cell sheet comprises cell adhesion proteins and cell junction proteins.

9. (canceled)

10. The method of claim 1, wherein the MSCs are isolated from the subepithelial layer of human umbilical cord tissue.

11. (canceled)

12. The method of claim 1, wherein the MSCs express a cytokine selected from the group consisting of hepatocyte growth factor (HGF), vascular endothelial growth factor (VEGF) and interleukin-10 (IL-10).

13. The method of claim 12, wherein expression of the cytokine in the cell sheet is increased relative to a suspension of MSCs containing an equivalent number of cells.

14. The method of claim 13, wherein the cell sheet expresses the cytokine for at least 10 days after transplantation to a tissue in a host organism.

15. The method of claim 1, wherein the cell sheet expresses extracellular matrix proteins and cell junction proteins for at least 10 days after transplantation to a tissue in a host organism.

16. The method of claim 15, wherein the extracellular matrix proteins are selected from the group consisting of fibronectin, laminin and collagen.

17. The method of claim 15, wherein the cell junction proteins are selected from the group consisting of Vinculin, Integrin-β1, Connexin 43, β-catenin, Integrin-linked kinase and N-cadherin.

18. The method of claim 1, wherein initial cell density of the MSCs in a cell culture support used to prepare the cell sheet is from 0.5×10⁴/cm² to 9×10⁵/cm².

19. The method of claim 1, wherein the MSCs do not express Human Leukocyte Antigen-DR isotype (HLA-DR), Human Leukocyte Antigen-DP isotype (HLA-DP), or Human Leukocyte Antigen-DQ isotype (HLA-DQ).

20. (canceled)

21. The method of claim 1, wherein the cell sheet remains attached to a tissue in a host organism for at least 10 days after transplantation to the tissue.

22. The method of claim 1, wherein the MSCs in the cell sheet are allogeneic to the subject.

23. The method of claim 1, wherein the subject is a human.

24-26. (canceled)

27. A method of preventing or reducing rupture of a uterine incision in a subject in need thereof, comprising applying a mesenchymal stem cell (MSC) sheet to the uterus of the subject, wherein the MSC sheet comprises one or more layers of aggregated confluent mesenchymal stem cells (MSCs), and wherein applying the MSC sheet to the uterus prevents or reduces rupture of the uterine incision relative to an incision in a uterus to which the MSC sheet is not applied.

28. The method of claim 1, wherein the MSC is a human umbilical cord mesenchymal stem cell (hUC-MSC).
